(19) United States
(12) Patent Application Publication
(10) Pub. No.: US 2022/0016168 A1
(43) Pub. Date: Jan. 20, 2022

(54) METHODS OF USING CD27 ANTIBODIES AS CONDITIONING TREATMENT FOR ADOPTIVE CELL THERAPY

(71) Applicant: Celldex Therapeutics, Inc., Hampton, NJ (US)

(72) Inventors: Lizhen HE, Allentown, PA (US); Tibor KELER, Pipersville, PA (US); Anna WASIUK, Easton, PA (US)

(21) Appl. No.: 17/298,071

(22) PCT Filed: Dec. 10, 2019
(86) PCT No.: PCT/US2019/065375

§ 371 Date: May 28, 2021

Related U.S. Application Data
(60) Provisional application No. 62/778,019, filed on Dec. 11, 2018.

(51) Int. Cl. A61K 35/17; C07K 16/28; A61P 35/00

(52) U.S. Cl. CPC A61K 35/17; A61P 35/00; C07K 16/2878

(57) ABSTRACT

Methods of performing adoptive immunotherapy comprising administering to patients a CD27 antibody in combination with an adoptive immunotherapy (e.g., genetically modified autogenous and allogenous T-cells) are provided.

### Related Applications

This application claims the benefit of priority of U.S. Provisional Application No. 62/778,019 (filed on Dec. 11, 2018). The contents of the aforementioned application is hereby incorporated by reference in its entireties.

### Background

Adoptive cell therapy (ACT) is quickly becoming a powerful tool to fight disease (e.g., cancer). In one form of ACT, T-cells are extracted from a patient, genetically modified, expanded in vitro, and returned to the same patient. In other instances, T-cells from a donor other than the patient receiving the cells are genetically modified, expanded and given to the patient. However, adoptively transferred T-cells fail to functionally persist in all patients and show generally poor efficacy in solid tumors. Currently chemo-drug cyclophosphamide (Cy) and fludarabine (Flu) are used to induce lymphopenia (create empty space and remove cytokine sinks) as conditioning treatment, so that the in vivo expansion and efficacy of ACT are enhanced. Nevertheless, these chemo-drugs induce reduction of pan-leukocytes resulting in neutropenia and other severe adverse effects.

Varlilumab is a fully human monoclonal antibody that uniquely binds to CD27 and has been shown to activate human T-cells in the context of T-cell receptor stimulation and to deplete cells that express high level of CD27 on the surface through effector functions, resulting in regulatory T-cell (Tre_{reg})-preferential lymphopenia. In addition, varlilumab is able to block the binding of human CD27 with human and mouse CD70, the unique natural ligand of CD27, thus depriving cells of this endogenous co-stimulatory signaling. Specifically, varlilumab is an antibody possessing properties of agonist, depleting and ligand blocking. In the case of CD27-expressing hematological malignancies, varlilumab may also provide direct therapeutic effects through effector functions. Thus, it is the object of the present disclosure to provide improved conditioning methods for ACT by using a CD27 antibody alone or in combination with other conditioning agents.

### SUMMARY

Provided herein are methods of conditioning treatment for ACT in a subject in need thereof comprising administering to a subject a CD27 antibody) and transferring engineered autogenic or allogenic immune cells (e.g., T-cells) to the subject.

In one aspect, the method comprises (in any order) administering a CD27 antibody to a subject to reduce the number and proliferation of endogenous T-cells and transferring engineered autogenic or allogenic T-cells to the subject, such that the transferred T-cells are preferentially expanded in the subject relative to a subject not treated with a CD27 antibody. In another aspect, a method of treating cancer in a subject in need thereof is provided, comprising (in any order) administering a CD27 antibody to the subject and transferring engineered autogenic or allogenic T-cells to the subject. In another aspect, a method of suppressing tumor growth in a subject in need thereof is provided, comprising (in any order) administering a CD27 antibody to the subject and transferring engineered autogenic or allogenic T-cells to the subject. In another aspect, a method of conditioning a subject for ACT is provided, comprising administering a CD27 antibody to the subject and transferring engineered autogenic or allogenic T-cells to the subject.

In one embodiment, the CD27 antibody is administered either simultaneously or before or after administration of ACT. In another embodiment, the CD27 antibody causes depletion of T_{reg}, CD4 helper T-cells (CD4-Th), and CD8 T-cells in the subject. In another embodiment, the CD27 antibody causes preferential (i.e., greater) depletion of T_{reg} compared to CD8 T-cells in the subject. In another embodiment, the CD27 antibody blocks CD27 interaction with CD70 in the subject.

In one embodiment, the transferred T-cells are ex vivo genetically engineered and comprise expanded T-cells or tumor-infiltrated lymphocytes (TILs). In another embodiment, the genetically engineered T-cells express a chimeric antigen receptor (CAR) or a T-cell receptor (TCR) which recognizes a tumor-associated antigen. In another embodiment, the transferred T-cells display an effector phenotype and function. In another embodiment, the transferred T-cells respond to antigen stimulation. In another embodiment, the transferred and expanded T-cells display antitumor activity.

In another embodiment, the genetically engineered T-cells express a mutated human CD27 receptor such that the transferred T-cells are not depleted by the CD27 antibody. In another embodiment, the human CD27 receptor comprises the mutation R87A, according to Kabat numbering (also referred to as R107A which includes the leader sequence) as set forth in SEQ ID NOs: 71 (without leader sequence) and 70 (with leader sequence). In another embodiment, the transferred T-cells carrying the CD27_{R87A} mutation can be co-stimulated by endogenous CD70 expressed on recipient's cells in the absence of competition of recipient's T-cells, in which the wild type CD27 is blocked by CD27 antibody conditioning treatment (polarized CD70 co-stimulation).

In one embodiment, the CD27 antibody is an agonist. In another embodiment, the CD27 antibody is an IgG1. In another embodiment, the CD27 antibody comprises CDRH1, CDRH2, and CDRH3 sequences comprising the amino acid sequences set forth in SEQ ID NOs: 5, 6, and 7, respectively, and CDRL1, CDRL2, and CDRL3 sequences comprising the amino acid sequences set forth in SEQ ID NOs: 8, 9, and 10, respectively. In another embodiment, the CD27 antibody comprises variable heavy and variable light chain amino acid sequences set forth in SEQ ID NO: 3 and SEQ ID NO: 4, respectively. In another embodiment, the CD27 antibody comprises heavy and light chain amino acid sequences set forth in SEQ ID NO: 68 and SEQ ID NO: 69, respectively. In another embodiment, the CD27 antibody is varlilumab, or an antibody that has same properties as varlilumab and/or which binds to the same epitope as varlilumab.

In one embodiment, patients are treated for cancers selected from the group consisting of leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma, marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

In one embodiment, the CD27 antibody is administered at least 12 hours before the T-cells are transferred. In another embodiment, the CD27 antibody is administered at least 24 hours before the T-cells are transferred. In another embodiment, the CD27 antibody is administered at least 48 hours before the T-cells are transferred. In another embodiment, the CD27 antibody is administered approximately 7 days (e.g., ±1 day) before and again approximately 2 days (e.g., ±1 day) before the T-cells are transferred. In another embodiment, the CD27 antibody is administered approximately 14 days (e.g., ±1 day) before and again approximately 2 days (e.g., ±1 day) before the T-cells are transferred. In another embodiment, the T-cells are administered by intravenous infusion.

In one embodiment, the CD27 antibody can be used in combination with current standard conditioning treatment, e.g. cyclophosphamide plus fludarabine. In another embodiment, the combination of CD27 antibody and cyclophosphamide plus fludarabine has synergetic effect as conditioning treatment for ACT.

In another aspect, use of a CD27 antibody as a conditioning agent in adoptive T-cell therapy is provided. In another aspect, a CD27 antibody for use as a conditioning agent in adoptive T-cell therapy is provided. In another aspect, a genetically engineered T-cell which expresses a mutated human CD27 (hCD27) receptor such that the engineered T-cell is activated by human CD70 but is significantly less depleted by a hCD27 antibody than recipient's T-cells is provided. In one embodiment, the mutated hCD27 receptor comprises a mutation that abolishes binding to a CD27 antibody. In another embodiment, the mutation reduces binding of varlilumab to the mutated hCD27 receptor. In another embodiment, the mutated hCD27 receptor comprises the mutation R87A as set forth in SEQ ID NO: 71.

Taken together, using varlilumab as conditioning treatment for ACT as described herein has at least five layers of advantages: 1) T_{reg} preferentially depleted lymphopenia; 2) skewed CD70 co-stimulation to genetically edited adoptively transferred cells (e.g., CD27_{R87A}) through the blocking of recipient's self CD70-CD27 interaction; 3) additional agonistic activities provided by varlilumab interaction with recipient's cells, resulting in the release of cytokines and chemokines, to promote the expansion and function of the transferred cells; and 4) no varlilumab-mediated depletion of adoptively transferred cells due to the CD27 mutation (e.g., CD27_{R87A}); 5) synergetic effect on ACT antitumor activity by combining with current conditioning treatment.

### BRIEF DESCRIPTION OF THE DRAWINGS

FIG. 1 shows the effect of varlilumab on decreasing total CD4 and CD8 T-cell populations in spleen and peripheral lymph nodes (pLNs) of human CD27 transgenic mice (hCD27^{+/+}mCD27^{wt}).

FIGS. 2A-2C show the effect of varlilumab on decreasing T_{reg} populations (CD4⁺Foxp3⁺) in hCD27^{+/+}mCD27^{wt} mice. FIG. 2A shows the percentage of T_{reg} out of total live cells and the absolute T_{reg} numbers in spleen and pLNs. FIG. 2B shows the ratios of CD8 T-cells and CD4Th-cells (CD4⁺Foxp3⁻) to T_{reg} (CD4⁺Foxp3⁺) in spleen and pLNs. FIG. 2C shows the CD27 expression levels in subsets of T-cells.

FIG. 3 shows adoptive T cell transfer schema.

FIGS. 4A and 4B show the optimization of varlilumab pretreatment for T cell depletion and donor cell expansion in recipient mice expressing human CD27 transgene and deficient in mouse CD27 due to gene knock-out (hCD27^{+/+}mCD27^{-/-}). FIG. 4A shows the depletion of recipient CD3 T-cells in the spleen and pLNs on the day for adoptive cell transfer and 14 days later following the injections of varlilumab or hIgG1 isotype control on the indicated days. FIG. 4B shows the 14 days in vivo expansion of donor CD8 T-cells in the spleen and pLNs of recipient mice following the indicated pretreatments.

FIG. 5 is representative histograms of carboxyfluorescein succinimidyl ester (CFSE) dilution in donor origin CD8 T-cells in spleen and pLNs of hCD27^{+/+}mCD27^{-/-} recipient mice pretreated with varlilumab or hIgG1, showing the increased donor cell expansion upon varlilumab pretreatmen.

FIG. 6 shows the increased donor origin CD8 T-cells at a 3-week time course in varlilumab-pretreated recipients compared to hIgG1 isotype control.

FIGS. 7A and 7B show a CD8 T-cell preferential expansion upon varlilumab pretreatment. FIG. 7A depicts dominant donor CD8 T-cell expansion upon CD3 T-cell transfer. FIG. 7B depicts greater magnitude of expansion upon CD8 T-cell transfer than CD4 T-cell transfer.

FIGS. 8A and 8B show the abolished or reduced expansion of donor CD8 T-cells that are lacking CD27 signaling through blocking CD70 or using mCD27^{-/-} donor cells. FIG. 8A shows the abolishment of CD8 T-cell expansion in varlilumab-pretreated hCD27^{+/+}mCD27^{-/-} recipient mice. FIG. 8B shows the reduction of CD8 T-cell expansion in Rag2^{-/-} recipient mice.

FIGS. 9A-9D show increased expansion of CD8 T-cells isolated from hCD27^{+/+}mCD27^{-/-} mice compared to CD8 T-cells from mCD27^{-/-} mice or mCD27^{wt} mice as donors following a mouse CD27 Ab AT124mG2a pretreatment. FIG. 9A depicts the expansion of donor cells with or without expressing mCD27 or hCD27. FIG. 9B depicts the depletion induced by AT124mG2a. FIG. 9C depicts no co-stimulatory effect triggered by AT124mG2a. FIG. 9D depicts no blocking effect of AT124mG2a on mCD70-mCD27 binding.

FIGS. 10A-10D show the reduced expansion of donor CD8 T-cells by dissecting the depleting and ligand blocking activities of CD27 Ab for conditioning treatment. FIG. 10A shows the expansion of donor origin CD8 T-cells in spleen and pLNs of hCD27^{+/+}mCD27^{-/-} recipient mice pretreated with varlilumab, 2C2, varliₘᵤₜ or hIgG1. FIG. 10B shows similar depleting activity of 2C2 and varlilumab but not varliₘᵤₜ. FIG. 10C shows similar co-stimulatory activity of 2C2 and varlilumab but not varliₘᵤₜ. FIG. 10D shows ligand blocking by varlilumab but not by 2C2.

FIGS. 11A-11C show the decreased expansion of donor CD8 T-cells in hCD27^{-/-} transgenic mice that express wild type mCD27 (hCD27^{+/+}mCD27^{wt}) upon varlilumab conditioning treatment relative to that in hCD27^{+/+}mCD27^{-/-} recipient mice. FIG. 11A is representative histograms of CFSE dilution in these two strains of recipient mice given varlilumab or hIgG1 pretreatment. FIG. 11B shows the expansion of donor cells in spleen and pLNs of these two strains of recipient mice given varlilumab or hIgG1 pretreatment. FIG. 11C shows the similar levels of T cell depletion in these two strains of recipient mice upon varlilumab pretreatment.

FIGS. 12A and 12B show suppressed proliferation of endogenous cells relative to donor cells in hCD27^{+/+}mCD27^{-/-} mice and to endogenous cells of hCD27^{+/+}mCD27^{wt} mice following varlilumab treatment. FIG. 12A shows percentage of Ki-67 in donor origin and recipient endogenous cells in these two strains of mice upon varlilumab pretreatment. FIG. 12B shows percentage of Ki-67 in endogenous cells of these two strains of mice upon varlilumab treatment without cell transfer.

FIGS. 13A and 13B show the increased antitumor efficacy of adoptive transferred OT-I T-cells following varlilumab pretreatment in E.G7 tumor model. FIG. 13A depicts tumor growth curves and Kaplan-Meier survival plot, comparing varlilumab treatment with or without OT-I cell transfusion plus or minus SIINFEKL peptide injection. FIG. 13B depicts tumor growth curves and Kaplan-Meier survival plot, showing higher survival rate in mice receiving varlilumab versus hIgG1 pretreatment for OT-I cell therapy in the settings of both presence and absence of peptide stimulation.

FIGS. 14A-14C show different profiles of recipients' cell depletion and extent of donor cell expansion after conditioning treatments with varlilumab versus Cy+Flu combination. FIG. 14A depicts absolute numbers of total and subpopulations of WBC per μl blood upon the indicated pretreatment on the day for cell transfer. FIG. 14B depicts recipients' T_{reg}-cell recovery 14 days post cell transfer. FIG. 14C depicts the donor cells expansion in blood, spleen and pLNs of recipients receiving the indicated pretreatment.

FIG. 15A and 15B show that varlilumab is superior to Cy+Flu as conditioning treatment for OT-I T-cells therapy in E.G7 tumor model. FIG. 15A depicts tumor growth curves. FIG. 15B depicts Kaplan-Meier survival plot.

FIG. 16 shows synergy of varlilumab and Cy+Flu as conditioning treatment for OT-I T-cells therapy in E.G7 tumor model.

FIGS. 17A and 17B show that human CD27_{R87A} mutation abolishes recognition of varlilumab and does not interrupt the binding with CD70 detected by ELISA (FIG. 17A) and ForteBio Octet system (FIG. 17B)

### DETAILED DESCRIPTION

As described herein, the invention is based on the discovery that CD27 antibodies reduce native T-cells (particularly T_{reg}) and promote the expansion of exogenously transferred T-cells. Accordingly, the present disclosure provides methods for the treatment of disease (e.g., cancer) comprising administering to a patient a CD27 antibody in combination with an adoptive immunotherapy (e.g., transfer of engineered autogenic or allogenic T-cells).

### A. Definitions

In order that the present description may be more readily understood, certain terms are first defined. Additional definitions are set forth throughout the detailed description. Unless defined otherwise, all technical and scientific terms used herein have the same meaning as commonly understood by one of ordinary skill in the art, and conventional methods of immunology, protein chemistry, biochemistry, recombinant DNA techniques, and pharmacology are employed.

As used herein, the term “adoptive immunotherapy” or “adoptive cell therapy” (ACT) refers to a process whereby autologous or allogeneic cells of various hematopoietic lineages (e.g., lymphocytes or T-cells) are transferred to a patient or subject to treat disease.

The term “adoptive T-cell therapy” refers to a process whereby autologous or allogeneic T-cells are transferred to a patient or subject to treat disease.

The term “T-cell receptor” or “TCR”, refers to a molecule found on the surface of T-cells, or T lymphocytes, that is responsible for recognizing fragments of antigen as peptides bound to major histocompatibility complex (MHC) molecules.

The term “CD27” (also referred to as “CD27 molecule”, “CD27L receptor”, “S1521”, “T-cell activation antigen CD27”, “TNFRSF7,” “MGC20393,” “tumor necrosis factor receptor superfamily, member 7”, “T-cell activation antigen S152” “Tp55”, “Tumor necrosis factor receptor superfamily member 7”, “CD27 antigen”, and “T-cell activation antigen CD27”) refers to a receptor that is a member of the TNF-receptor superfamily, which binds to ligand CD70. CD27 is required for generation and long-term maintenance of T-cell immunity and plays a key role in regulating B-cell activation and immunoglobulin synthesis. The term “CD27” includes any variants or isoforms of CD27 which are naturally expressed by cells (e.g., human CD27 deposited with GENBANK® having accession no. AAH12160.1). Accordingly, CD27 antibodies may cross-react with CD27 from species other than human. Alternatively, the CD27 antibodies may be specific for human CD27 and may not exhibit any cross-reactivity with other species. CD27 or any variants and isoforms thereof, may either be isolated from cells or tissues that naturally express them or be recombinantly produced using well-known techniques in the art and/or those described herein. Preferably the CD27 antibodies are targeted to human CD27 which has a normal glycosylation pattern.

Genbank® (Accession No. AAH12160.1) reports the amino acid sequence of human CD27 as follows (SEQ ID NO: 1):

MARPHPWWLC VLGTLVGLSA TPAPKSCPER HYWAQGKLCC
QMCEPGTFLV KDCDQHRKAA QCDPCIPGVS FSPDHHTRPH
CESCRHCNSG LLVRNCTITA NAECACRNGW QCRDKECTEC
DPLPNPSLTA RSSQALSPHP QPTHLPYVSE MLEARTAGHM
QTLADFRQLP ARTLSTHWPP QRSLCSSDFI RILVIFSGMF
LVFTLAGALF LHQRRKYRSN KGESPVEPAE PCRYSCPREE
EGSTIPIQED YRKPEPACSP

The term “CD70” (also referred to as “CD70 molecule”, “CD27L”, “CD27LG”, “TNFSF7,” “tumor necrosis factor (ligand) superfamily member 7,” “CD27 ligand,” “CD70 antigen,” “surface antigen CD70,” “tumor necrosis factor ligand superfamily, member 7,” “Ki-24 antigen,” and “CD27-L”) refers to the ligand for CD27 (see, for example, Bowman M R et al., J. Immunol. 1994 Feb. 15; 152 (4):1756-61). CD70 is a type II transmembrane protein that belongs to the tumor necrosis factor (TNF) ligand family It is a surface antigen on activated T and B lymphocytes that induces proliferation of co-stimulated T-cells, enhances the generation of cytolytic T-cells, and contributes to T-cell activation. It has also been suggested that CD70 plays a role in regulating B-cell activation and immunoglobulin synthesis, and cytotoxic function of natural killer cells (Hintzen R Q et al., J. Immunol. 1994 Feb. 15; 152 (4):1762-73).

Genbank® (Accession No. NP_001243) reports the amino acid sequence of human CD70 as follows (SEQ ID NO: 2):

MPEEGSGCSV RRRPYGCVLR AALVPLVAGL VICLVVCIQR
FAQAQQQLPL ESLGWDVAEL QLNHTGPQQD PRLYWQGGPA
LGRSFLHGPE LDKGQLRIHR DGIYMVHIQV TLAICSSTTA
SRHHPTTLAV GICSPASRSI SLLRLSFHQG CTIASQRLTP
LARGDTLCTN LTGTLLPSRN TDETFFGVQW VRP

The term “antibody” as referred to herein includes whole antibodies and any antigen binding fragment (i.e., “antigen-binding portion”) or single chain thereof. An “antibody” refers, in one preferred embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In certain embodiments, the numbering of amino acid positions in the antibodies described herein (e.g., amino acid residues in the Fc region) and identification of regions of interest, e.g., CDRs, use the Kabat system (Kabat, E. A., et al. (1991) Sequences of Proteins of Immunological Interest, Fifth Edition, U.S. Department of Health and Human Services, NIH Publication No. 91-3242). Other embodiments described herein may define CDRs using the Chothia numbering system (Chothia et al. (1989) Nature 342:877-883). Thomas et al. [(1996) Mol Immunol 33 (17/18):1389-1401] exemplifies the identification of CDR boundaries according to Kabat and Chothia definitions. Other embodiments described herein may define CDRs using the IMGT numbering system (Lefranc et al, Dev. Comp. Immunol 2005; 29 (3):185-203). Other embodiments described herein may define CDRs using the AHo numbering system (Honegger and Pluckthun, J. Mol. Biol. 2001; 309 (3):657-70).

An immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In certain embodiments, the antibodies described herein are of the IgG1 or IgG2 subtype. Immunoglobulins, e.g., IgG1, exist in several allotypes, which differ from each other in at most a few amino acids. “Antibody” includes, by way of example, both naturally occurring and non-naturally occurring antibodies; monoclonal and polyclonal antibodies; chimeric and humanized antibodies; human and nonhuman antibodies; wholly synthetic antibodies; and single chain antibodies.

The term “antigen-binding portion” of an antibody (or simply “antibody portion”), as used herein, refers to one or more fragments of an antibody that retain the ability to specifically bind to an antigen (e.g., human CD27). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The term “monoclonal antibody,” as used herein, refers to an antibody which displays a single binding specificity and affinity for a particular epitope. Accordingly, the term “human monoclonal antibody” refers to an antibody which displays a single binding specificity and which has variable and optional constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23 (9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368 (6474): 856-859); Lonberg, N. (1994) Handbook of Experimental Pharmacology 113:49-101; Lonberg, N. and Huszar, D. (1995) Intern. Rev. Immunol. Vol. 13: 65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., humanized antibodies).

As used herein, a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “isolated antibody,” as used herein, is intended to refer to an antibody which is substantially free of other antibodies having different antigenic specificities (e.g., an isolated antibody that specifically binds to human CD27 is substantially free of antibodies that specifically bind antigens other than human CD27). An isolated antibody that specifically binds to an epitope of may, however, have cross-reactivity to other CD27 proteins from different species. However, the antibody preferably always binds to human CD27. In addition, an isolated antibody is typically substantially free of other cellular material and/or chemicals. In one embodiment of the invention, a combination of “isolated” antibodies having different CD27 specificities is combined in a well-defined composition.

The terms “specific binding,” “selective binding,” “selectively binds,” and “specifically binds,” refer to antibody binding to an epitope on a predetermined antigen. Typically, the antibody binds with an equilibrium dissociation constant (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 2000 instrument using recombinant human CD27 as the analyte and the antibody as the ligand and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

The term “K_{D},” as used herein, is intended to refer to the dissociation equilibrium constant of a particular antibody-antigen interaction. Typically, the human antibodies of the invention bind to CD27 with a dissociation equilibrium constant (K_{D}) of approximately 10⁻⁸ M or less, such as less than 10⁻⁹ M or 10⁻¹⁰ M or even lower when determined by surface plasmon resonance (SPR) technology in a BIACORE 2000 instrument using recombinant human CD27 as the analyte and the antibody as the ligand. Other methods for determining K_{D} include equilibrium binding to live cells expressing CD27 via flow cytometry (FACS) or in solution using KinExA® technology.

The term “ka” as used herein, is intended to refer to the on rate constant for the association of an antibody with the antigen.

The term “epitope” or “antigenic determinant” refers to a site on an antigen to which an immunoglobulin or antibody specifically binds. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. Methods for determining what epitopes are bound by a given antibody (i.e., epitope mapping) are well known in the art and include, for example, immunoblotting and immunoprecipitation assays, wherein overlapping or contiguous peptides are tested for reactivity with a given antibody. Methods of determining spatial conformation of epitopes include techniques in the art, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)). The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on CD27” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target.

Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc ; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25 (1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding polypeptides, antibodies, or antibody fragments (e.g., V_{H}, V_{L}, CDR3), is intended to refer to a nucleic acid molecule in which the nucleotide sequences are essentially free of other genomic nucleotide sequences, e.g., those encoding other sequences may naturally flank the nucleic acid in human genomic DNA.

The term, “pharmaceutically acceptable,” as used herein, refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

The terms “cancer” and “cancerous,” as used herein, refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma and various types of head and neck cancer.

The terms “treat,” “treating,” and “treatment,” as used herein, refer to therapeutic measures described herein. The methods of treatment employ administration to a subject (such as a human) the combination disclosed herein in order to cure, delay, reduce the severity of, or ameliorate one or more symptoms of the disease or disorder or recurring disease or disorder, or in order to prolong the survival of a subject beyond that expected in the absence of such treatment.

The terms “inhibitors” and “antagonists,” or “activators” and “agonists,” as used herein, refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a compound that reduces, blocks, or inactivates a constitutive activity.

The term “inhibition” or “inhibit” as used herein, refers to any statistically significant decrease in biological activity, including partial and full blocking of the activity. For example, “inhibition” can refer to a statistically significant decrease of about 10%, about 20%, about 30%, about 40%, about 50%, about 60%, about 70%, about 80%, about 90%, about 95%, about 96%, about 97%, about 98%, about 99%, or about 100% in biological activity.

The term “inhibits growth” as used herein, of a tumor includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or about 100%.

The terms “expansion” or “expand” as used herein, refers to an increase in the number of immune cells (e.g., number of transferred T-cells). For example, “expansion” can refer to a statistically significant increase of at least 10%, at least 20%, at least 30%, at least 40%, at least at least 50%, at least 60%, at least 70%, at least 80%, at least 90%, at least 95%, at least 100% (i.e., at least 2 fold), at least 3 fold, at least 5 fold or at least 10 fold. An increase in the number of immune cells (e.g., T-cells) can be measured as an increase in the total number of cells or as a percentage of total immune cells (e.g., total T-cell population).

The terms “depletion” or “deplete” as used herein, refers to a decrease in the number of immune cells (e.g., number of T_{reg}). For example, “depletion” can refer to a statistically significant decrease of about 10%, about 20%, about 30%, about 40%, about 50%, about 60%, about 70%, about 80%, about 90%, about 95%, about 96%, about 97%, about 98%, about 99%, or about 100% in cell number as compared to control or cell number prior to treatment. A decrease in cell number can be measured as a decrease in the total number of cells or as a percentage of total immune cells (e.g., total T-cell population).

The term “therapeutic agent” in intended to encompass any and all compounds that have an ability to decrease or inhibit the severity of the symptoms of a disease or disorder, or increase the frequency and/or duration of symptom-free or symptom-reduced periods in a disease or disorder, or inhibit or prevent impairment or disability due to a disease or disorder affliction, or inhibit or delay progression of a disease or disorder, or inhibit or delay onset of a disease or disorder, or inhibit or prevent infection in an infectious disease or disorder. Non-limiting examples of therapeutic agents include small organic molecules, monoclonal antibodies, bispecific antibodies, recombinantly engineered biologics, RNAi compounds, and commercial antibodies.

As used herein, “administering” refers to the physical introduction of a composition comprising a therapeutic agent to a subject, using any of the various methods and delivery systems known to those skilled in the art. Exemplary routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “effective dose” or “effective dosage” is defined as an amount sufficient to achieve or at least partially achieve the desired effect. The term “therapeutically effective dose” or “therapeutically effective amount” is defined as an amount sufficient to cure or at least partially arrest the disease and its complications in a patient already suffering from the disease. Amounts effective for this use will depend upon the severity of the disorder being treated and the general state of the patient's own immune system.

The term “patient” includes human and other mammalian subjects that receive either prophylactic or therapeutic treatment.

The term “subject” includes any mammal. For example, the methods and compositions herein disclosed can be used to treat a subject having cancer. In a particular embodiment, the subject is a human.

The term “immune cell” is a cell of hematopoietic origin and that plays a role in the immune response. Immune cells include lymphocytes (e.g., B cells and T-cells), natural killer cells, and myeloid cells (e.g., monocytes, macrophages, eosinophils, mast cells, basophils, and granulocytes).

The term “T-cell” refers to a CD4 T-cell or a CD8 T-cell. The term T-cell encompasses TH1 cells, TH2 cells and TH17 cells.

The term “T_{reg}” or “regulatory T-cells” refers to a specialized population of T-cells which are able to suppress the activation and expansion of other T-cells to maintain a fine homeostatic balance between reactivity to foreign- and self-antigens. These T_{reg} are characterized by a high level expression of surface interleukin-2 receptor a chain (CD25) and an intracellular expression of a master switch transcription factor called forkhead box protein P3 (Foxp3).

The term “T-cell-mediated response” refers to any response mediated by T-cells, including effector T-cells (e.g., CD8 cells) and helper T-cells (e.g., CD4 cells). T-cell mediated responses include, for example, T-cell cytotoxicity and proliferation.

The term “cytotoxic T lymphocyte (CTL) response” refers to an immune response induced by cytotoxic T-cells. CTL responses are mediated primarily by CD8 T-cells.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune response or reaction includes, e.g., activation or inhibition of a T-cell, e.g., an effector T-cell or a Th cell, such as a CD4 or CD8 T-cell, or the inhibition of a T_{reg} cell.

An “immunomodulator” or “immunoregulator” refers to an agent, that may be involved in modulating, regulating, or modifying an immune response. “Modulating,” “regulating,” or “modifying” an immune response refers to any alteration in a cell of the immune system or in the activity of such cell (e.g., an effector T-cell). Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments, the immunomodulator is located on the surface of a T-cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

As used herein, the term “chimeric antigen receptor” or “CAR” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain. The “chimeric antigen receptor (CAR)” is sometimes called a “chimeric receptor”, a “T-body”, or a “chimeric immune receptor (CIR).”

The phrase “CAR ligand” used interchangeably with “CAR antigen” means any natural or synthetic molecule (e.g. small molecule, protein, peptide, lipid, carbohydrate, nucleic acid) or part or fragment thereof that can specifically bind to the CAR. The “intracellular signaling domain” means any oligopeptide or polypeptide domain known to function to transmit a signal causing activation or inhibition of a biological process in a cell, for example, activation of an immune cell such as a T-cell or a NK cell. Examples include ILR chain, CD28 and/or 003ζ.

As used herein, the phase “CAR T-cell” refers to a chimeric antigen receptor-expressing T-cell. These cells are typically derived from a patient with a disease or condition and genetically modified in vitro to express at least one CAR with an arbitrary specificity to a ligand (e.g., a cancer antigen). The cells perform at least one effector function (e.g. induction of cytokines) that is stimulated or induced by the specific binding of the ligand to the CAR and that is useful for treatment of the same patient's disease or condition. The T-cells can be, e.g., cytotoxic T-cells or helper T-cells.

As used herein, “cancer antigen” refers to (i) tumor- specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a”, “an” and “the” include plural referents unless the context clearly dictates otherwise. The use of “or” or “and” means “and/or” unless stated otherwise. Furthermore, use of the term “including” as well as other forms, such as “include”, “includes”, and “included”, is not limiting.

The term “about” as used herein when referring to a measurable value such as an amount, a temporal duration and the like, is encompasses variations of up to ±10% from the specified value. Unless otherwise indicated, all numbers expressing quantities of ingredients, properties such as molecular weight, reaction conditions, etc., used herein are to be understood as being modified by the term “about”.

### B. CD27 Antibodies

As described in further detail herein, the invention generally involves the use of CD27 antibodies in combination with an adoptive cell therapy (e.g., transfer of exogenous T-cells).

In one embodiment, the CD27 antibody (e.g., monoclonal antibody) is a full-length antibody or antibody binding fragment thereof. CD27 antibodies (or VH/VL domains derived therefrom) suitable for use in the invention can be generated using methods known in the art. Alternatively, art recognized CD27 antibodies (or antibody fragments thereof) can be used. Antibodies that bind to the same epitope and/or compete with any of the art-recognized antibodies for binding to CD27 also can be used.

An exemplary CD27 antibody is 1F5 (varlilumab). Varlilumab is a fully human monoclonal antibody that uniquely binds to CD27 and has been shown to activate human T-cells in the context of T-cell receptor stimulation and may also provide direct therapeutic effects against tumors that express CD27, and is described in WO 2011/130434, the teachings of which are hereby expressly incorporated by reference. In one embodiment, the antibody comprises the heavy and light chain CDRs or variable regions of varlilumab. Accordingly, in one embodiment, the antibody comprises the CDR1, CDR2, and CDR3 domains of the VH region of varlilumab having the sequence set forth in SEQ ID NO: 3, and the CDR1, CDR2, and CDR3 domains of the VL region of varlilumab having the sequence set forth in SEQ ID NO: 4. In another embodiment, the antibody comprises heavy chain CDR1, CDR2 and CDR3 domains having the sequences set forth in SEQ ID NOs: 5, 6, and 7, respectively, and light chain CDR1, CDR2 and CDR3 domains having the sequences set forth in SEQ ID NOs: 8, 9, and 10, respectively. In another embodiment, the antibody comprises a VH region having the amino acid sequence set forth in SEQ ID NO: 3. In another embodiment, the antibody comprises a VL region having the amino acid sequence set forth in SEQ ID NO: 4. In another embodiment, the antibody comprises VH and VL regions having the amino acid sequences set forth in SEQ ID NO: 3 and SEQ ID NO: 4, respectively. In another embodiment, the antibody comprises a heavy chain having the amino acid sequence set forth in SEQ ID NO: 68. In another embodiment, the antibody comprises a light chain having the amino acid sequence set forth in SEQ ID NO: 69. In another embodiment, the antibody comprises heavy and light chains having the amino acid sequences set forth in SEQ ID NO: 68 and SEQ ID NO: 69, respectively.

The full-length heavy and light chain sequences of varlilumab are as follows:

Heavy chain (SEQ ID NO: 68):
QVQLVESGGG VVQPGRSLRL SCAASGFTFS SYDMHWVRQA PGKGLEWVAV
IWYDGSNKYY ADSVKGRFTI SRDNSKNTLY LQMNSLRAED TAVYYCARGS
GNWGFFDYWG QGTLVTVSSA STKGPSVFPL APSSKSTSGG TAALGCLVKD
YFPEPVTVSW NSGALTSGVH TFPAVLQSSG LYSLSSVVTV PSSSLGTQTY
ICNVNHKPSN TKVDKKVEPK SCDKTHTCPP CPAPELLGGP SVFLFPPKPK
DTLMISRTPE VTCVVVDVSH EDPEVKFNWY VDGVEVHNAK TKPREEQYNS
TYRVVSVLTV LHQDWLNGKE YKCKVSNKAL PAPIEKTISK AKGQPREPQV
YTLPPSRDEL TKNQVSLTCL VKGFYPSDIA VEWESNGQPE NNYKTTPPVL
DSDGSFFLYS KLTVDKSRWQ QGNVFSCSVM HEALHNHYTQ KSLSLSPGKG SS
Light chain (SEQ ID NO: 69):
DIQMTQSPSS LSASVGDRVT ITCRASQGIS RWLAWYQQKP EKAPKSLIYA
ASSLQSGVPS RFSGSGSGTD FTLTISSLQP EDFATYYCQQ YNTYPRTFGQ
GTKVEIKRTV AAPSVFIFPP SDEQLKSGTA SVVCLLNNFY PREAKVQWKV
DNALQSGNSQ ESVTEQDSKD STYSLSSTLT LSKADYEKHK VYACEVTHQG
LSSPVTKSFN RGEC

Other examples of CD27 antibodies include 2C2, 3H12, 2G9, 1H8, 3A10, 3H8, and 1G5, also described further in WO 2011/130434. Accordingly, in one embodiment, the antibody comprises the CDR1, CDR2, and CDR3 domains of the VH region of 2C2 having the sequence set forth in SEQ ID NO: 12, and the CDR1, CDR2, and CDR3 domains of the VL region of 2C2 having the sequence set forth in SEQ ID NO: 13. In another embodiment, the antibody comprises the CDR1, CDR2, and CDR3 domains of the VH region of 3H12 having the sequence set forth in SEQ ID NO: 14, and the CDR1, CDR2, and CDR3 domains of the VL region of 3H12 having the sequence set forth in SEQ ID NO: 15. In another embodiment, the antibody comprises the CDR1, CDR2, and CDR3 domains of the VH region of 2G9 having the sequence set forth in SEQ ID NO: 16, and the CDR1, CDR2, and CDR3 domains of the VL region of 2G9 having the sequence set forth in SEQ ID NO: 17. In another embodiment, the antibody comprises the CDR1, CDR2, and CDR3 domains of the VH region of 1H8 having the sequence set forth in SEQ ID NO: 18, and the CDR1, CDR2, and CDR3 domains of the VL region of 1H8 having the sequence set forth in SEQ ID NO: 19. In another embodiment, the antibody comprises the CDR1, CDR2, and CDR3 domains of the VH region of 3A10 having the sequence set forth in SEQ ID NO: 20, and the CDR1, CDR2, and CDR3 domains of the VL region of 3A10 having the sequence set forth in SEQ ID NO: 21. In another embodiment, the antibody comprises the CDR1, CDR2, and CDR3 domains of the VH region of 3H8 having the sequence set forth in SEQ ID NO: 22, and the CDR1, CDR2, and CDR3 domains of the VL region of 3H8 having the sequence set forth in SEQ ID NO: 23. In another embodiment, the antibody comprises the CDR1, CDR2, and CDR3 domains of the VH region of 1G5 having the sequence set forth in SEQ ID NO: 24, and the CDR1, CDR2, and CDR3 domains of the VL region of 1G5 having the sequence set forth in SEQ ID NO: 25.

The exact boundaries of CDRs can be defined differently according to different methods. In some embodiments, the CDRs of the V_{H} and V_{L} regions are defined according to the Kabat numbering system. In another embodiment, the CDRs of the V_{H} and V_{L} regions are defined according to the IMGT numbering system. In another embodiment, the CDRs of the V_{H} and V_{L} regions are defined according to Chothia numbering system. In another embodiment, the CDRs of the V_{H} and V_{L} regions are defined according to the AHo numbering system.

In another embodiment, the antibody comprises the V_{H} and V_{L} CDR sequences of 2C2 (shown in SEQ ID NOs: 26-28 and 29-31, respectively). In another embodiment, the antibody comprises the V_{H} and V_{L} CDR sequences of 3H12 (shown in SEQ ID NOs: 32-34 and 35-37, respectively). In another embodiment, the antibody comprises the V_{H} and V_{L} CDR sequences of 2G9 (shown in SEQ ID NOs: 38-40 and 41-43, respectively). In another embodiment, the antibody comprises the V_{H} and V_{L} CDR sequences of 1H8 (shown in SEQ ID NOs: 44-46 and 47-49, respectively). In another embodiment, the antibody comprises the V_{H} and V_{L} CDR sequences of 3A10 (shown in SEQ ID NOs: 50-52 and 53-55, respectively). In another embodiment, the antibody comprises the V_{H} and V_{L} CDR sequences of 3H8 (shown in SEQ ID NOs: 56-58 and 60-61, respectively). In another embodiment, the antibody comprises the V_{H} and V_{L} CDR sequences of 1G5 (shown in SEQ ID NOs: 62-64 and 65-67, respectively). Each of the above-referenced CDRs are present in the same relative order as they are present in the corresponding antibody.

Sequences substantially homologous (e.g., at least 80%, 90%, 95%, 98% or 99% identical to the aforementioned sequences) are also encompassed.

Other examples of CD27 antibodies include the antibodies C2177, C2191, C2192 and C2186 described in U.S. Pat. No. 9,102,737; antibody hCD27.15 described in U.S. Pat. No. 9,527,916; antibodies described in U.S. Pat. No. 8,481,029; and multimeric CD27 antibodies described in U.S. patent application Ser. No: 15/557,035.

In another embodiment, the CD27 antibody is an antibody that competes for binding with, and/or binds to the same epitope on CD27 as, the antibodies described herein. In another embodiment, these antibodies are characterized by activation of the CD27 receptor. Activation of the CD27 receptor can be measured by any suitable means in the art. Assays to evaluate the effects of the antibodies on functional properties of CD27 (e.g., ligand binding, T-cell proliferation, cytokine production) are described e.g., in WO 2011/130434, which is incorporated herein by reference thereto.

CD27 antibodies for use in the methods described herein can be full-length, for example, any of the following isotypes: IgG1, IgG2, IgG3, IgG4, IgM, IgA1, IgA2, IgAsec, IgD, and IgE. Alternatively, the CD27 antibodies can be fragments such as an antigen-binding portion or a single chain antibody (e.g., a Fab, F(ab′)₂, Fv, a single chain Fv fragment, an isolated complementarity determining region (CDR) or a combination of two or more isolated CDRs). The CD27 antibodies can be any kind of antibody, including, but not limited to, human, humanized, and chimeric antibodies.

In another embodiment, the CD27 antibody may be modified to enhance or diminish its interactions with host effector systems or to reduce adverse side effects. In another embodiment, the CD27 antibodies also can be linked to a second molecule (e.g., as a bispecific molecule) having a binding specificity which is different from the antibody, such as proteins expressed on T-cells (e.g., CD3, CD25, CD137, CD154), or an Fc receptor (e.g., FcγRI (CD64), FcγRIIA (CD32), FcγRIIB1 (CD32), FcγRIIB2 (CD32), FcγRIIIA (CD16a), FcγRIIIB (CD16b), FcεRI, FcεRII (CD23), FcαRI (CD89), Fcα/μR, and FcRn), or an NK receptor (e.g. CD56), or proteins expressed on B cells (e.g. CD19, CD20). In another embodiment, the CD27 antibody can be linked to a second molecule that binds a protein on T-cells, such as OX-40, 41BB, CD28, ICOS, CD40L, GITR, TIM1, CD30, HVEM, LIGHT, SLAM, DR3, CD2, CD226, PD-1, CTLA4, LAG3, CD160, BTLA, VISTA, LAIR1, TIM3, 2B4 or TIGIT.

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BlAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627.

Preferably, CD27 antibodies bind to CD27 with high affinity, for example, with a K_{D} of 10⁻⁷ M or less, 10⁻⁸ M or less, 10⁻⁹ M or less, 10⁻¹⁰ M or less, 10⁻¹¹ M or less, 10⁻¹² M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, 10⁻⁷ M to 10⁻¹² M, 10⁻⁸ M to 10⁻¹² M, 10⁻⁹ M to 10−12 M, 10⁻¹⁰ M to 10⁻¹² M, 10⁻¹¹ M to 10⁻¹² M. Alternative, CD27 antibodies bind to CD27 with a Ka of 10⁺⁷ M⁻¹ or greater, 10⁺⁸ M⁻¹ or greater, 10⁺⁹ M⁻¹ or greater, 10⁺¹⁰ M⁻¹ or greater, 10⁺¹¹ M⁻¹ or greater, 10⁺¹² M⁻¹ or greater, 10⁺⁷ M⁻¹ to 10⁺¹² M⁻¹, 10⁺⁸ M⁻¹ to 10⁺¹²M⁻¹, 10⁺⁹ M⁻¹ to 10⁺¹² M⁻¹, 10⁺¹⁰ M⁻¹ to 10⁺¹²M⁻¹, 10⁺¹¹ M⁻¹ to 10⁺¹² M⁻¹, 10⁺¹² M⁻¹ to 10⁺⁷ M⁻¹, 10⁺¹¹ M⁻¹ to 10⁺⁷ M⁻¹, 10⁺¹⁰M⁻¹ to 10⁺⁷ M⁻¹, or 10⁺⁹ M⁻¹ to 10⁺⁷ M⁻¹. In a particular embodiment, the CD27 antibody binds to human CD27 with an equilibrium dissociation constant KD of 10⁻⁹ M or less, or alternatively, an equilibrium association constant Ka of 10⁺⁹M⁻¹ or greater as measured by Biacore analysis.

In another embodiment, CD27 antibodies are not native antibodies or are not naturally-occurring antibodies. For example, CD27 antibodies have post-translational modifications that are different from those of antibodies that are naturally occurring, such as by having more, less or a different type of post-translational modification.

The CD27 antibody can be administered to the patient by any route suitable for the effective delivery to the patient. For example, many small molecule inhibitors are suitable for oral administration. Antibodies and other biologic agents typically are administered parenterally, e.g., intravenously, intraperitoneally, subcutaneously or intramuscularly.

In other embodiments, the CD27 antibody has at least one of the following features:
1) reduces endogenous T_{reg} (CD4+Foxp3⁺) population
2) blocks CD27 interacting with CD70
3) induces CD27-mediated lymphopenia
4) promotes survival of adoptively transferred immune cells (e.g., T-cells)
5) promotes proliferation/expansion of adoptively transferred immune cells (e.g., T-cells)
6) suppresses the proliferation and/or survival of endogenous T-cells
7) enhances antitumor efficacy of an adoptive immunotherapy treatment (e.g., exogenously transferred T-cells).

Accordingly, a CD27 antibody that exhibits one or more of these functional properties (e.g., biochemical, immunochemical, cellular, physiological or other biological activities, or the like) as determined according to methodologies known to the art and described herein, will be understood to relate to a statistically significant difference in the particular activity relative to that seen in the absence of the antibody (e.g., or when a control antibody of irrelevant specificity is present). Preferably, CD27 antibody-induced increases in a measured parameter (e.g., T-cell proliferation, cytokine production) effects a statistically significant increase by at least 10% of the measured parameter, more preferably by at least 20%, 30%, 40%, 50%, 60%, 70%, 80%, 90%, 95%, 100% (i.e., 2 fold), 3 fold, 5 fold or 10 fold, and in certain preferred embodiments, an antibody described herein may increase the measured parameter by greater than 92%, 94%, 95%, 97%, 98%, 99%, 100% (i.e., 2 fold), 3 fold, 5 fold or 10 fold. Conversely, CD27 antibody-induced decreases in a measured parameter (e.g., tumor volume, tumor growth, or T_{reg} number) effects a statistically significant decrease by at least 10% of the measured parameter, more preferably by at least 20%, 30%, 40%, 50%, 60%, 70%, 80% or 90%, and in certain preferred embodiments, an antibody described herein may decrease the measured parameter by greater than 92%, 94%, 95%, 97%, 98% or 99%.

In another embodiment, the CD27 antibody reduces total endogenous T_{reg} by about 10%, about 20%, about 30%, about 40%, about 50%, about 60%, about 70%, about 80%, about 90%, about 95%, about 96%, about 97%, about 98%, about 99%, or about 100% as compared to control or number of T_{reg} prior to treatment.

In another embodiment, the CD27 antibody promotes survival and/or expansion of adoptively transferred immune cells (e.g., T-cells). In particular, the CD27 antibody promotes survival and/or expansion of adoptively transferred T-cells by at least 10%, at least 20%, at least 30%, at least 40%, at least at least 50%, at least 60%, at least 70%, at least 80%, at least 90%, at least 95%, at least 100% (i.e., at least 2 fold), at least 3 fold, at least 5 fold or at least 10 fold as compared to control (see, e.g., Examples 1-7 below).

In another embodiment, the CD27 antibody is formulated in a pharmaceutical composition, e.g., a composition comprising one or a combination of CD27 antibodies as described herein, formulated together with a carrier (e.g., a pharmaceutically acceptable carrier). The pharmaceutical compositions also can be administered in combination therapy, i.e., combined with other agents. For example, the combination therapy can include a composition of the present invention with at least one or more additional therapeutic agents, such as anti-inflammatory agents, DMARDs (disease-modifying anti-rheumatic drugs), immunoregulatory agents, and chemotherapeutics. The pharmaceutical compositions described herein can also be administered in conjunction with radiation therapy. Co-administration of the pharmaceutical compositions with other antibodies are also encompassed by the methods described herein.

As used herein, the terms “carrier” and “pharmaceutically acceptable carrier” includes any and all solvents, salts, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., CD27 antibody, may be coated in a material to protect the compound from the action of acids and other natural conditions that may inactivate the compound.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the antibodies of the invention may be administered once or twice weekly by subcutaneous or intramuscular injection or once or twice monthly by subcutaneous or intramuscular injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

A composition as described herein can be administered by a variety of methods known in the art. As will be appreciated by the skilled artisan, the route and/or mode of administration will vary depending upon the desired results. The active compounds can be prepared with carriers that will protect the compound against rapid release. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Regardless of the route of administration selected, the compounds of the present invention, which may be used in a suitable hydrated form, and/or the pharmaceutical compositions described herein, are formulated into pharmaceutically acceptable dosage forms by conventional methods known to those of skill in the art.

### C. Adoptive Immunotherapy

Adoptive immunotherapy or adoptive cell therapy (ACT) refers to the infusion into patients of autologous or allogeneic cells of various hematopoietic lineages to treat disease (see, e.g., WO 2016133907). One category of adoptive immunotherapy is hematopoietic stem cell (“HSC”) transplantation. HSC involves the infusion of autologous or allogeneic stem cells to reestablish hematopoietic function in patients whose bone marrow or immune system is damaged or defective. The HSCs may be genetically modified, for example to treat congenital genetic diseases. Another category of adoptive immunotherapy is T-cell immunotherapy or adoptive T-cell therapy, which involves the infusion of autologous or allogeneic T lymphocytes that are selected and/or engineered ex vivo to target specific antigens, typically tumor-associated antigens (see, e.g., WO 2016133907 A1). The T lymphocytes are typically obtained from the peripheral blood of the donor by leukapheresis. In some T-cell immunotherapy methods, the T lymphocytes obtained from the donor, such as tumor infiltrating lymphocytes (“TIL”s), are expanded in culture and selected for antigen specificity without altering their native specificity (Stevanovic et al, J. Clin. Oncol., EPub ahead of print, 10.1200/JCO.2014.58.9093 (2015); Dudley et al., J. Clin. Oncol. 23 (10):2346-2357 (2005)).

In other T-cell immunotherapy methods, T lymphocytes obtained from the donor are engineered ex vivo, typically by transduction with viral expression vectors, to express chimeric antigen receptors (“CAR”s) of predetermined specificity. Chimeric antigen receptors (CARs) are genetically-engineered, artificial transmembrane receptors, which confer an arbitrary specificity for a ligand onto an immune effector cell (e.g. a T-cell, natural killer cell or other immune cell) and which results in activation of the effector cell upon recognition and binding to the ligand. Typically these receptors are used to impart the antigen specificity of a monoclonal antibody onto a T-cell. For example, CARs typically include an extracellular domain, such as the binding domain from a scFv, that confers specificity for a desired antigen; a transmembrane domain; and one or more intracellular domains that trigger T-cell effector functions, such as the intracellular domain from CD3 or FcRy, and, optionally, one or more co-stimulatory domains drawn, e.g., from CD28 and/or 4- IBB (Jensen and Riddell, Immunological Reviews 257: 127-144 (2014)).

The main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors. Moreover, a new generation of CARs containing a binding domain, a hinge, a transmembrane and the signaling domain derived from CD3 or FcRy together with one or more co-stimulatory signaling domains (e.g., intracellular co-stimulatory domains derived from CD28, CD137, CD134 and CD278) has been shown to more effectively direct antitumor activity as well as increased cytokine secretion, lytic activity, survival and proliferation in CAR expressing T-cells in vitro, in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

Chimeric antigen receptor-expressing effector cells (e.g. CAR T-cells) are cells that are typically derived from a patient with a disease or condition and genetically modified in vitro to express at least one CAR with an arbitrary specificity to a ligand. The cells perform at least one effector function (e.g., induction of cytokines) that is stimulated or induced by the specific binding of the ligand to the CAR and that is useful for treatment of the same patient's disease or condition. The effector cells may be T-cells (e.g. cytotoxic T-cells or helper T-cells). One skilled in the art would understand that other cell types (e.g. a natural killer cell or a stem cell) may express CARs and that a chimeric antigen receptor effector cell may comprise an effector cell other than a T-cell. The effector cell may be a cell (e.g. a cytotoxic T-cell) that exerts its effector function (e.g. a cytotoxic T-cell response) on a target cell when brought in contact or in proximity to the target or target cell (e.g. a cancer cell) (see e.g., Chang and Chen (2017) Trends Mol Med 23 (5):430-450).

In still other T-cell immunotherapy methods, T lymphocytes obtained from the donor are engineered ex vivo, typically by transduction with viral expression vectors, to express T-cell receptors (“TCR”s) that confer desired specificity for antigen presented in the context of specific HLA alleles (Liddy et al, Nat. Med. 18 (6):980-988 (2012)).

In certain embodiments, the adoptive immunotherapy comprises administering a genetically engineered T-cell which expresses a mutated human CD27 (hCD27) receptor. The mutation can be produced by adding, substituting, or deleting an amino acid at one or more positions, such that a CD27 antibody has reduced affinity for the hCD27 receptor. The mutation can be either conservative or non-conservative.

The mutation can be produced using known techniques, such as CRISPR (Clustered Regularly Interspaced Short Palindromic Repeats) technology (e.g., CRISPR/Cas9). In one embodiment, the engineered T-cell is activated by human CD70, but is significantly less depleted by an anti-hCD27 antibody than native T-cells. In another embodiment, the mutated hCD27 receptor comprises a mutation that reduces binding to a CD27 antibody. In another embodiment, the mutation reduces binding of varlilumab to hCD27 receptor. Specifically, the binding of varlilumab and CD70 to CD27 can be differentiated by introducing a single mutation into CD27, i.e., CD70 but not varlilumab recognizes CD27 carrying R87A mutation (CD27R87A). Thus, in a particular embodiment, the mutated hCD27 receptor comprises the mutation of arginine at position 87 to alanine (R87A) as shown in SEQ ID NO: 71 (also shown at position 107 in SEQ ID NO: 70; R107A).

In adoptive T-cell therapy methods, T-cells are typically obtained from the peripheral blood of the donor. It is often desirable to obtain as many T-cells as possible from the donor, in order to increase the likelihood of obtaining T lymphocytes of desired antigen specificity and/or phenotype (Jensen and Riddell, Immunological Reviews 257: 127-144 (2014)). Accordingly, the donor may be treated with a mobilizing agent in order to effect release of T-cells resident in the bone marrow and other physiological niches into the peripheral circulation.

In one embodiment, the CD27 antibody is administered at least 12 hours before the T-cells are transferred. In another embodiment, the CD27 antibody is administered at least 24 hours before the T-cells are transferred. In another embodiment, the CD27 antibody is administered at least 48 hours before the T-cells are transferred. In another embodiment, the CD27 antibody is administered at least 72 hours before the T-cells are transferred. In another embodiment, the CD27 antibody is administered at least 4 days before the T-cells are transferred. In another embodiment, the CD27 antibody is administered at least 5 days before the T-cells are transferred. In another embodiment, the CD27 antibody is administered at least 6 days before the T-cells are transferred. In another embodiment, the CD27 antibody is administered at least 7 days before the T-cells are transferred.

In another embodiment, the CD27 antibody is administered at least twice before the T-cells are transferred. In another embodiment, the CD27 antibody is administered up to 14 days before and again approximately 2 days before the T-cells are transferred. In another embodiment, the T-cells are administered by intravenous infusion.

### D. Combination Therapies

Methods of performing adoptive immunotherapy and combination therapies described herein may also be used in conjunction with other therapies for treating cancer (i.e., anti-cancer agents).

For example, the methods and therapies described herein can be used in combination (e.g., simultaneously or separately) with one or more standard treatments, such as chemotherapy (e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo21/TRAIL (a 6× combo)), one or more proteasome inhibitors (e.g., bortezomib or MG132), one or more Bc1-2 inhibitors (e.g., BH3I-2′ (bc1-x1 inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), anti-angiogenic agents targeting VEGFR2 (e.g., Cyramza™/ramucirumab), synthetic triterpenoids (see Hyer et al., Cancer Research 2005; 65:4799-808), c-FLIP (cellular FLICE-inhibitory protein) modulators (e.g., natural and synthetic ligands of PPARγ (peroxisome proliferator-activated receptor γ), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3β inhibitors, IAP inhibitors and/or genotoxic drugs.

The methods of performing adoptive immunotherapy and combination therapies described herein can further be used in combination with one or more anti-proliferative cytotoxic agents. Classes of compounds that may be used as anti-proliferative cytotoxic agents include, but are not limited to, the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with CD27 antibodies and the adoptive immunotherapies described herein include, without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone B1, -dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with the adoptive immunotherapies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX™, can also be administered to the patient. When employing the methods or compositions described herein, other agents used in the modulation of tumor growth or metastasis in a clinical setting, such as antimimetics, can also be administered as desired.

Methods for safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

Methods of performing adoptive immunotherapy described herein may also be used in conjunction with one or more immunotherapies to upregulate or stimulate an immune response. For example, the CD27 antibodies and adoptive immunotherapies described herein can be used in combination (e.g., simultaneously or separately) with one or more immunoregulatory agents (e.g., immunostimulatory agents or checkpoint inhibitors). Immunoregulatory agents include small molecule drugs, antibodies or antigen binding portions thereof, and/or protein ligands that are effective in stimulating immune responses to thereby further enhance, stimulate or upregulate immune responses in a patient.

In one embodiment, the immunoregulatory agent is (i) an agonist of a stimulatory (e.g., co-stimulatory) molecule (e.g., receptor or ligand) and/or (ii) an antagonist of an inhibitory signal or molecule (e.g., receptor or ligand) on immune cells, such as T-cells. In either case, the agonistic or antagonistic molecule results in amplifying immune responses, such as antigen-specific T-cell responses. For example, collectively, these molecules may be called immunoregulatory agents. In certain aspects, an immunoregulatory agent is enhances innate immunity, e.g., by acting as (i) an agonist of a stimulatory (including a co-stimulatory) molecule (e.g., receptor or ligand) or (ii) an antagonist of an inhibitory (including a co-inhibitory) molecule (e.g., receptor or ligand) on cells involved in innate immunity, e.g., NK cells.

As described above, T-cell responses can be stimulated by administering an antagonist (inhibitor or blocking agent) of a protein that inhibits T-cell activation. Such inhibitors are often called immune checkpoint inhibitors. For example, potential targets for checkpoint inhibitors include CTLA-4, PD-1, PD-L1, PD-L2, and LAG-3, and any of the following proteins: TIM-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, TIM-1, and TIM-4. Exemplary immune checkpoint inhibitors include Opdivo™ (nivolumab/BMS-936558) (to PD-1), Yervoy™ (ipilimumab) or Tremelimumab (to CTLA-4), Tecentriq™ (atezolizmab) (to PD-L1), Durvalumab (to PD-L1), Bavencio™ (Avelumab) (to PD-L1), and Pembrolizumab/MK-3475 (to PD-1).

Alternatively, T-cell responses can be stimulated by administering an agonist of a protein that stimulates T-cell activation, such as B7-1, B7-2, CD28, 4-1BB (CD137), 4-1BBL, ICOS, ICOS-L, 0X40, OX40L, CD70, CD27, CD40, DR3 and CD28H.

Other targets for immnoregulation include members of the immunoglobulin super family (IgSF). For example, the CD27 antibodies and adoptive immunotherapies, e.g., described herein, may be administered (simultaneously or sequentially) to a subject with an agent that targets a member of the IgSF family to increase an immune response. For example, a CD27 antibody or adoptive immunotherapy may be administered with an agent that targets (or binds specifically to) a member of the B7 family of membrane-bound ligands or a member of the TNF and TNFR family of molecules (ligands or receptors). For example, members of the B7 family of molecules may include, but is not limited to, B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor binding specifically to a B7 family member. Examples of the TNF and TNFR family of molecules (ligands or receptors) may include, but is not limited to, CD40 and CD40L, OX-40, OX-40L, CD70, CD27L, CD30, CD30L, 4-1BBL, CD137, TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDA1, EDA2, TNFR1, Lymphotoxin α/TNFβ, TNFR2, TNFα, LTβR, Lymphotoxin α 1β32, FAS, FASL, RELT, DR6, TROY, and NGFR (see, e.g., Tansey (2009) Drug Discovery Today 00:1).

Other exemplary agents that modulate one of the above proteins and may be used in combination with the present invention to treat cancer (e.g., lung cancer), include: galiximab (to B7.1), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), or pidilizumab (to PD-1).

Other agents that can be combined include antagonists of inhibitory receptors on NK cells or agonists of activating receptors on NK cells, e.g., an antagonists of KIR (e.g., lirilumab); antagonists of cytokines that inhibit T-cell activation or agonists of cytokines that stimulate T-cell activation; antagonists (or inhibitors or blocking agents) of proteins of the IgSF family or B7 family or the TNF family that inhibit T-cell activation or antagonists of cytokines that inhibit T-cell activation (e.g., IL-6, IL-10, TGF-β, VEGF; “immunosuppressive cytokines”); and/or agonists of stimulatory receptors of the IgSF family, B7 family or the TNF family or of cytokines that stimulate T-cell activation, for stimulating an immune response.

Other agents that can be combined include those that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

Other agents that can be combined include those that inhibit TGF-β signaling.

Other agents that can be combined include those that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Other agents that can be combined include those that deplete or block T_{reg} cells, e.g., an agent that specifically binds to CD25.

Other agents that can be combined include those that inhibit a metabolic enzyme such as indoleamine dioxigenase (IDO), dioxigenase, arginase, or nitric oxide synthetase.

Other agents that can be combined include those that inhibit the formation of adenosine or inhibit the adenosine A2A receptor.

Other agents that can be combined include those that reverse/prevent T-cell anergy or exhaustion and therapies that trigger an innate immune activation and/or inflammation at a tumor site.

Other agents that can be combined include immunoregulatory agents, and may be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking T_{reg} or other immune suppressing cells; a therapy that stimulates positive immune regulation, e.g., with agonists that stimulate the CD-137, OX-40, and/or GITR pathway and/or stimulate T-cell effector function; a therapy that increases systemically the frequency of anti-tumor T-cells; a therapy that depletes or inhibits T_{reg}, such as T_{reg} in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T-cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T-cell anergy or exhaustion; a therapy that triggers an innate immune activation and/or inflammation at a tumor site; administration of immune stimulatory cytokines; or blocking of immuno repressive cytokines.

Other agents that can be combined include one or more of agonistic agents that ligate positive costimulatory receptors, blocking agents that attenuate signaling through inhibitory receptors, antagonists, and one or more agents that increase systemically the frequency of anti-tumor T-cells, agents that overcome distinct immune suppressive pathways within the tumor microenvironment (e.g., block inhibitory receptor engagement (e.g., PD-L1/PD-1 interactions), deplete or inhibit T_{reg} (e.g., using an anti-CD25 monoclonal antibody (e.g., daclizumab) or by ex vivo anti-CD25 bead depletion), inhibit metabolic enzymes such as IDO, or reverse/prevent T-cell anergy or exhaustion) and agents that trigger innate immune activation and/or inflammation at tumor sites.

In certain embodiments, the nucleotide sequences of any of the above mentioned immunoregulatory agents may be administered to a patient using gene therapy techniques known in the art.

The immunotherapies and/or chemotherapeutic agent(s) can be administered according to therapeutic protocols well known in the art. It will be apparent to those skilled in the art that the administration of the chemotherapeutic agent(s) and/or immunotherapies can be varied depending on the disease being treated and the known effects of the chemotherapeutic agent(s) and/or immunotherapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

Other agents that can be combined include adjuvants. Examples of adjuvants which may be used with the CD27 antibodies of the present invention include: Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatised polysaccharides; polyphosphazenes; biodegradable microspheres; cytokines, such as GM-CSF, interleukin-2, -7, -12, and other like factors; 3D-MPL; CpG oligonucleotide; and monophosphoryl lipid A, for example 3-de-O-acylated monophosphoryl lipid A.

Further alternative adjuvants include, for example, saponins, such as Quil A, or derivatives thereof, including QS21 and QS7 (Aquila Biopharmaceuticals Inc., Framingham, Mass.); Escin; Digitonin; or Gypsophila or Chenopodium quinoa saponins; Montanide ISA 720 (Seppic, France); SAF (Chiron, California, United States); ISCOMS (CSL), MF-59 (Chiron); the SBAS series of adjuvants (e.g., SBAS-2 or SBAS-4, available from SmithKline Beecham, Rixensart, Belgium); Detox (Enhanzyn™) (Corixa, Hamilton, Mont.); RC-529 (Corixa, Hamilton, Mont.) and other aminoalkyl glucosaminide 4-phosphates (AGPs); polyoxyethylene ether adjuvants such as those described in WO 99/52549A1; synthetic imidazoquinolines such as imiquimod [S-26308, R-837], (Harrison, et al., Vaccine 19: 1820-1826, 2001; and resiquimod [S-28463, R-848] (Vasilakos, et al., Cellular immunology 204: 64-74, 2000; Schiff bases of carbonyls and amines that are constitutively expressed on antigen presenting cell and T-cell surfaces, such as tucaresol (Rhodes, J. et al., Nature 377: 71-75, 1995); cytokine, chemokine and co-stimulatory molecules as either protein or peptide, including for example pro-inflammatory cytokines such as Interferon, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L; immunostimulatory agents; endotoxin, [LPS], (Beutler, B., Current Opinion in Microbiology 3: 23-30, 2000); ligands that trigger Toll receptors to produce Th1-inducing cytokines, such as synthetic Mycobacterial lipoproteins, Mycobacterial protein p19, peptidoglycan, teichoic acid and lipid A; and CT (cholera toxin, subunits A and B) and LT (heat labile enterotoxin from E. coli, subunits A and B), heat shock protein family (HSPs), and LLO (listeriolysin O; WO 01/72329). These and various further Toll-like Receptor (TLR) agonists are described for example in Kanzler et al, Nature Medicine, May 2007, Vol 13, No 5. A preferred immunostimulatory agent for use in combination with a CD27 antibody of the invention is a TLR3 agonist, such as Poly IC.

Other agents that can be combined include vaccines. A vaccine can enhance the immune response against a vaccine antigen, for example a tumor antigen (to thereby enhance the immune response against the tumor) or an antigen from an infectious disease pathogen (to thereby enhance the immune response against the infectious disease pathogen). In certain embodiments, the methods comprise simultaneously or sequentially administering a CD27 antibody, a vaccine, and an adoptive immunotherapy (in any order).

A vaccine antigen can comprise, for example, an antigen or antigenic composition capable of eliciting an immune response against a tumor or against an infectious disease pathogen such as a virus, a bacteria, a parasite or a fungus. The antigen or antigens can be, for example, peptides/proteins, polysaccharides and/or lipids. The antigen or antigens be derived from tumors, such as the various tumor antigens previously disclosed herein. Alternatively, the antigen or antigens can be derived from pathogens such as viruses, bacteria, parasites and/or fungi, such as the various pathogen antigens previously disclosed herein. Additional examples of suitable tumor or pathogen antigens include, but are not limited to, the following:

Tumor-specific antigens, including tumor-specific membrane antigens, tumor-associated antigens, including tumor-associated membrane antigens, embryonic antigens on tumors, growth factor receptors, growth factor ligands, and any other type of antigen that is associated with cancer. A tumor antigen may be, for example, an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. For example, the antigen may include a tumor antigen, such as βhCG, gp100 or Pme117, CEA, gp100, TRP-2, NY-BR-1, NY-CO-58, MN (gp250), idiotype, Tyrosinase, Telomerase, SSX2, MUC-1, MAGE-A3, and high molecular weight-melanoma associated antigen (HMW-MAA) MART1, melan-A, EGFRvIII, NY-ESO-1, MAGE-1, MAGE-3, WT1, Her2,or mesothelin. Other antigens employed by the present invention (e.g., in a vaccine, used in combination with a CD27 antibody of the invention) include antigens from infectious disease pathogens, such as viruses, bacteria, parasites and fungi, examples of which are disclosed herein.

Viral antigens or antigenic determinants can be derived from, for example: Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreSl, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992,10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial antigens or antigenic determinants can be derived from, for example: Bacillus spp., including B. anthracis (eg botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R. rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, Pi1C, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (eg capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25,337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasitic/fungal antigens or antigenic determinants can be derived from, for example: Babesia spp., including B. microti; Candida spp., including C. albicans; Cryptococcus spp., including C. neoformans; Entamoeba spp., including E. histolytica; Giardia spp., including G. lamblia; Leshmania spp., including L. major; Plasmodium. faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.); Pneumocystis spp., including P. ;carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

It will be appreciated that in accordance with this aspect of the present invention antigens and antigenic determinants can be used in many different forms. For example, antigens or antigenic determinants can be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner Alternatively, antigens or antigenic determinants may be generated in situ in the subject by use of a polynucleotide coding for an antigen or antigenic determinant (as in so-called “DNA vaccination”), although it will be appreciated that the polynucleotides which can be used with this approach are not limited to DNA, and may also include RNA and modified polynucleotides as discussed above.

In one embodiment, a vaccine antigen can also be targeted, for example to particular cell types or to particular tissues. For example, the vaccine antigen can be targeted to Antigen Presenting Cells (APCs), for example by use of agents such as antibodies targeted to APC-surface receptors such as DEC-205, for example as discussed in WO 2009/061996 (Celldex Therapeutics, Inc), or the Mannose Receptor (CD206) for example as discussed in WO 03040169 (Medarex, Inc.).

Preferred routes of administration for vaccines include, for example, injection (e.g., subcutaneous, intravenous, parenteral, intraperitoneal, intrathecal). The injection can be in a bolus or a continuous infusion. Other routes of administration include oral administration.

### E. Indications

Exemplary diseases and conditions which can be treated by the methods described herein include, but are not limited to, proliferative disorders (e.g., cancer), HIV, Hepatitis C, immune disorders (e.g., autoimmune disorders), bacterial infections, fungal infections, parasitic infections, viral infections, and the like.

Examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to non-Hodgkin lymphoma and variants thereof, peripheral T-cell lymphomas, adult T-cell leukemia/lymphoma (ATL), cutaneous T-cell lymphoma (CTCL), large granular lymphocytic leukemia (LGF), Hodgkin's disease and Reed-Sternberg disease.

Other cancers which can be treated by the disclosed methods include, but are not limited to, myeloblasts promyelocyte myelomonocytic monocytic erythroleukemia, mantle cell lymphoma, primary central nervous system lymphoma, Burkitt's lymphoma and marginal zone B cell lymphoma, Polycythemia vera Lymphoma, multiple myeloma, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

In one embodiment, the patient has evidence of recurrent or persistent disease following primary chemotherapy. In another embodiment, the patient has had at least one prior platinum based chemotherapy regimen for management of primary or recurrent disease. In another embodiment, the patient has a cancer that is platinum-resistant or refractory. In another embodiment, the patient has evidence of recurrent or persistent disease following a) primary treatment or b) an adjuvant treatment. In another embodiment, the patient has a cancer that has become or been determined to be resistant to an immunoregulatory agent. For example, the patient has evidence of recurrent or persistent disease following treatment with a checkpoint inhibitor (e.g., ipilimumab, nivolumab, pembrolizumab, durvalumab, or atezolizumab).

In another embodiment, the patient has an advanced cancer. In one embodiment, the term “advanced” cancer denotes a cancer above Stage II. In another, “advanced” refers to a stage of disease where chemotherapy is typically recommended, which is any one of the following: 1. in the setting of recurrent disease: any stage or grade; 2. stage IC or higher, any grade; 3. stage IA or IB, grade 2 or 3; or 4. in the setting of incomplete surgery or suspected residual disease after surgery (where further surgery cannot be performed): any stage or grade.

It will be appreciated by those skilled in the art that reference herein to treatment extends to prophylaxis as well as the treatment of the noted proliferative disorders and symptoms.

Other disease indications the present methods can be used to treat include graft rejection, allergy and autoimmune diseases. Exemplary autoimmune diseases include, but are not limited to, multiple sclerosis, rheumatoid arthritis, type 1 diabetes, psoriasis, Crohn's disease and other inflammatory bowel diseases such as ulcerative colitis, systemic lupus eythematosus (SLE), autoimmune encephalomyelitis, myasthenia gravis (MG), Hashimoto's thyroiditis, Goodpasture's syndrome, pemphigus, Graves disease, autoimmune hemolytic anemia, autoimmune thrombocytopenic purpura, scleroderma with anti- collagen antibodies, mixed connective tissue disease, polypyositis, pernicious anemia, idiopathic Addison's disease, autoimmune associated infertility, glomerulonephritis, crescentic glomerulonephritis, proliferative glomerulonephritis, bullous pemphigoid, Sjogren's syndrome, psoriatic arthritis, insulin resistance, autoimmune diabetes mellitus, autoimmune hepatitis, autoimmune hemophilia, autoimmune lymphoproliferative syndrome (ALPS), autoimmune hepatitis, autoimmune hemophilia, autoimmune lymphoproliferative syndrome, autoimmune uveoretinitis, Guillain-Bare syndrome, arteriosclerosis and Alzheimer's disease. Exemplary allergic disorders include, but are not limited to allergic conjunctivitis, vernal conjunctivitis, vernal keratoconjunctivitis, and giant papillary conjunctivitis; nasal allergic disorders, including allergic rhinitis and sinusitis; otic allergic disorders, including eustachian tube itching; allergic disorders of the upper and lower airways, including intrinsic and extrinsic asthma; allergic disorders of the skin, including dermatitis, eczema and urticaria; and allergic disorders of the gastrointestinal tract.

In one embodiment, a method of treating an autoimmune disease in a subject in need thereof, comprising administering (simultaneously or sequentially) to a subject a CD27 antibody in combination with an adoptive T-cell therapy is provided. It will be appreciated by those skilled in the art that reference herein to treatment extends to prophylaxis as well as the treatment of the noted infections and symptoms.

Because viral infections are cleared primarily by T-cells, an increase in T-cell activity is therapeutically useful in situations where more rapid or thorough clearance of an infective viral agent would be beneficial to an animal or human subject. Recently, CAR T-cell therapy has been investigated for its usefulness in treating viral infections, such as human immunodeficiency virus (HIV), as described in PCT Publication No. WO 2015/077789; Hale et al., (2017) Engineering HIV-Resistant, Anti-HIV Chimeric Antigen Receptor T-Cells. Molecular Therapy, Vol. 25 (3): 570-579; Liu et al., (2016). ABSTRACT. Journal of Virology, 90 (21), 9712-9724; Liu et al., (2015). ABSTRACT. Journal of Virology, 89 (13), 6685-6694; Sahu et al., (2013). Virology, 446 (1-2), 268-275.

Thus, in one embodiment the CD27 antibodies and adoptive immunotherapies are administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. In another embodiment, the CD27 antibodies and adoptive immunotherapies are administered to treat systemic viral diseases, including, but not limited to, AIDS, influenza, the common cold, or encephalitis. In another embodiment, a method of treating a viral infection in a subject in need thereof, comprising administering (simultaneously or sequentially) to a subject a CD27 antibody in combination with an adoptive T-cell therapy is provided.

Other conditions the present methods can be used to treat include bacterial, fungal, and parasitic infectious diseases. As described above, the CD27 antibody and adoptive immunotherapy can be administered (simultaneously or sequentially) in combination with a vaccine which enhances the immune response against the vaccine antigen to treat the infection.

All references cited throughout this application, for example patent documents including issued or granted patents or equivalents; patent application publications; and non-patent literature documents or other source material; are hereby incorporated by reference herein in their entireties, as though individually incorporated by reference. Any sequence listing and sequence listing information is considered part of the disclosure herewith.

Those skilled in the art will recognize, or be able to ascertain using no more than routine experimentation, many equivalents of the specific embodiments described herein.

Such equivalents are intended to be encompassed by the following claims. Any combination of the embodiments disclosed in the any plurality of the dependent claims or Examples is contemplated to be within the scope of the disclosure.

The following examples are merely illustrative and should not be construed as limiting the scope of this disclosure in any way as many variations and equivalents will become apparent to those skilled in the art upon reading the present disclosure.

### V. EXAMPLES

### Example 1: Targeting CD27 with Varlilumab Leads to T-Cell Depletion

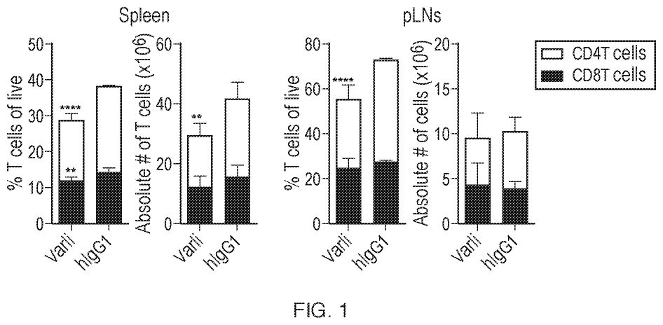

Human CD27 transgenic mice (hCD27^{+/-}mCD27^{wt}) were injected intraperitoneal (i.p.) with 0.2 mg of varlilumab or isotype control. Spleens and pLNs were collected 7 days later and processed for flow cytometry analysis. Results are displayed in FIG. 1, which shows percentage of CD8 and CD4 T-cells out of total live cells and their absolute numbers depicted in stacked bars. Data are from one representative experiment of two performed (n=3 mice per group), **p<0.01, ****p<0.0001; indicating that total T-cells, especially CD4 T-cells, are reduced in varlilumab-treated mice.

### Example 2: CD27-Mediated T-Cell Depletion is Preferentially on T_{reg}

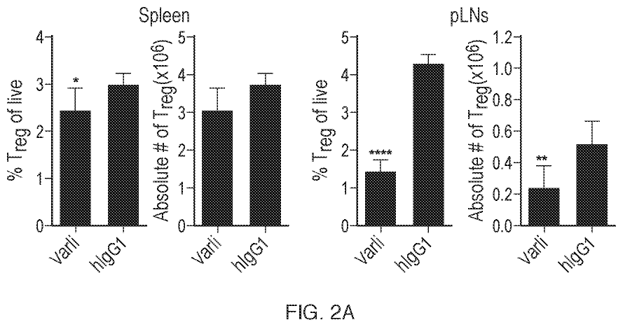

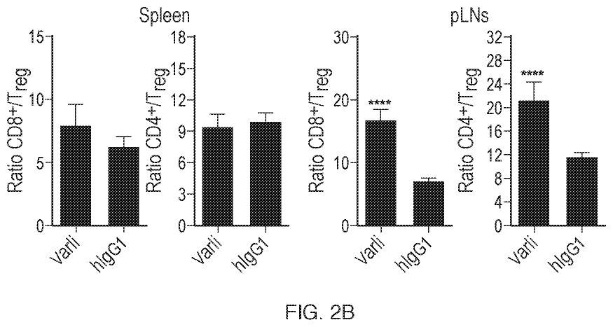

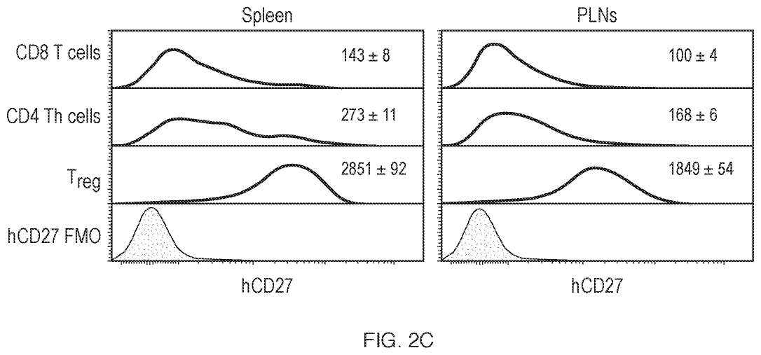

The same preparations of splenocytes and pLNs cells as in Example 1 were stained for CD8, CD4 and Foxp3. Results are displayed in FIG. 2. FIG. 2A shows the percentage of T_{reg} (CD4⁺Foxp3⁺) out of total live cells and their absolute numbers in both spleen and pLNs, and FIG. 2B shows the ratios of CD8 T-cells and CD4-Th (CD4⁺Foxp3⁻) to T_{reg} (CD4⁺Foxp3⁺) in both spleen and pLNs. FIG. 2C shows the expression of CD27 on these subsets of T-cells.

Data are from one representative experiment of two performed (n=3 mice per group), *p<0.05, **p<0.01, ****p<0.0001; indicating that percentage and absolute numbers of T_{reg} in peripheral lymph organs, especially in pLNs, are reduced in varlilumab-treated mice, resulting in the increase in ratios of CD8 T-cells to T_{reg} and CD4-Th to T_{reg}. Together, Examples 1 and 2 show that varlilumab-induced T-cell deletion is preferentially on T_{reg} and secondarily on CD4-Th, while the least reduction is on CD8 T-cells. This depletion pattern correlates with CD27 expression levels among these subsets of T-cells.

### Example 3: The Adoptive T Cell Transfer Schema

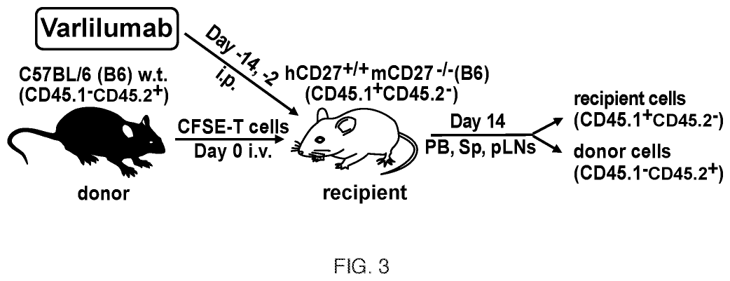

FIG. 3 shows the experimental design in all the studies except modifications specified in individual figure legends.

### Example 4: Optimal Treatment with Varlilumab Leads to a Remarkable Expansion of Adoptively Transferred CD8 T-Cells

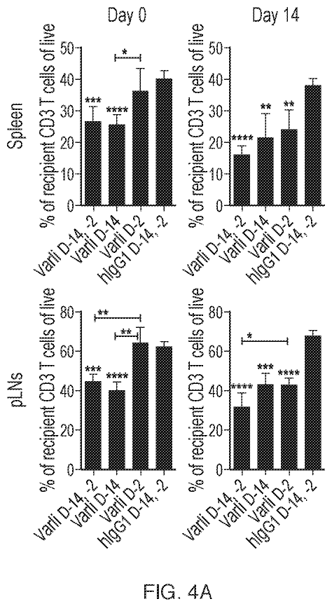

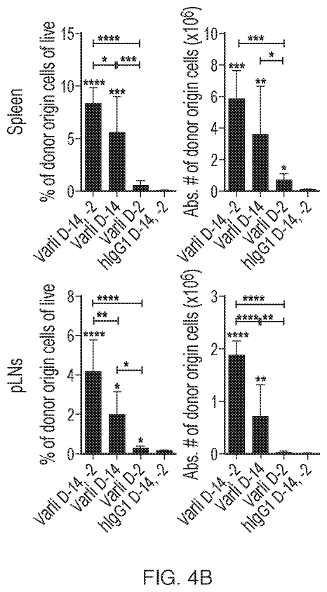

Two sets of hCD27^{+/+}mCD27^{-/-} mice were injected i.p. with 300 μg of varlilumab or hIgG1 isotype control on days −14 or −2 or both. Spleens and pLNs were collected from one set of mice on day 0 without cell transfusion and from another set of mice 14 days post intravenous (i.v.) transfusion with 2×10⁶ CFSE-labeled w.t. CD8 T-cells for the assessment of recipient cell depletion (FIG. 4A) and donor cell expansion (FIG. 4B). Percentages of recipient CD3 T-cells (CD45.1⁺CD45.2⁻CD3⁺) and donor origin CD8 T-cells (CD45.1⁻CD45.2⁺CD3⁺CD8⁺) out of total live cells in spleens and pLNs were determined by flow cytometry and the absolute numbers of donor origin cells were calculated based on total cell counts of spleens and pLNs.

Data are from one representative experiment of two performed (n=4 mice per group), *p<0.05, **p<0.01, ***p<0.001, ****p<0.0001, indicating statistical significance compared to isotype control or between groups as specified by the horizontal line. It is demonstrated that the greatest expansion was achieved in mice received varlilumab on day −14 and −2, median level of expansion was in mice received Ab on day −14 only and minimal expansion was in mice received Ab on day −2 only (FIG. 4B). Notably, recipient CD3 T-cell counts were reduced to the same extent in mice received 2 doses of varlilumab on day −14 and −2 versus 1 dose on day −14, and no significant reduction yet in mice injected with the Ab on day −2, compared to isotype control, on day 0 measurement (FIG. 4A). By day 14, compared to day 0, recipient CD3 T-cells counts were further decreased in mice that received varlilumab on day −14 and −2 or day −2 only but remained the same in mice injected with the Ab on day −14 only, suggesting that CD27-mediated T-cell depletion is a slow process and long-lasting status, and a severe T-cell reduction on the day of transfusion is crucial for the enhancement of donor cell expansion.

### Example 5: Representative Histogram of CFSE Dilution in Gated Donor-Origin CD8 T-Cells

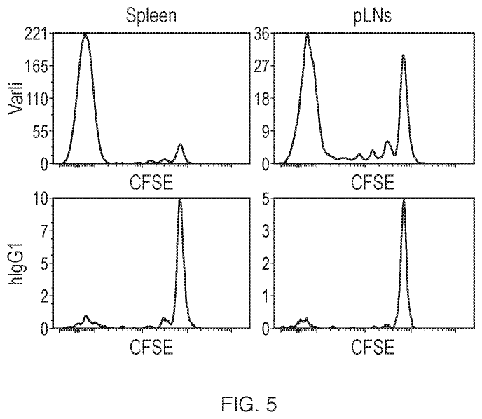

FIG. 5 shows representative flow cytometry histograms of CFSE dilution from the study as described in Example 4, FIG. 4B with varlilumab or hIgG1 isotype control pretreatment on day −14 and −2. The peak on right (high CFSE fluorescence) were transfused cells without going through division, whereas multiple peaks to the left (low CFSE fluorescence), seeing prominently in varlilumab pretreated mice but not in hIgG1 pretreated mice, were transfused cells having gone through multiple divisions. These histograms demonstrate that the increase in donor-origin CD8 T-cells in varlilumab-pretreated recipients is indeed due to enhanced proliferation.

### Example 6: Varlilumab Pretreatment Leads to a Long-Lasting Expansion of Adoptively Transferred Cells

hCD27^{+/+}mCD27^{-/-} mice were injected with 200 μg of varlilumab or hIgG1 on day −7 and −2. These mice were then transfused with 2×10⁶ CFSE-labeled CD8 T-cells on day 0. Spleens and pLNs were collected on day 7, 14 and 21 and processed for flow cytometry analysis. Data are displayed as the percentage of donor-origin CD8 T-cells out of total live cells in these lymphoid organs, n=3 mice per group, **p<0.01, ***p<0.001 in FIG. 6. Notations above bars indicate statistical significance compared to hIgG1. Horizontal lines indicate statistical significance between the groups specified. The results show a long-lasting accumulation of donor-origin CD8 T-cells in varlilumab-pretreated recipient mice compared to a much smaller and rapidly decreased same cell population in hIgGl-pretreated mice.

### Example 7: Varlilumab Pretreatment Favors the Expansion of CD8 T-Cells Over CD4 T-Cells

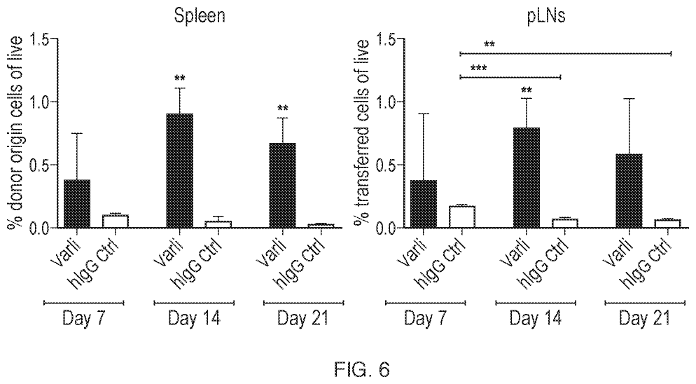

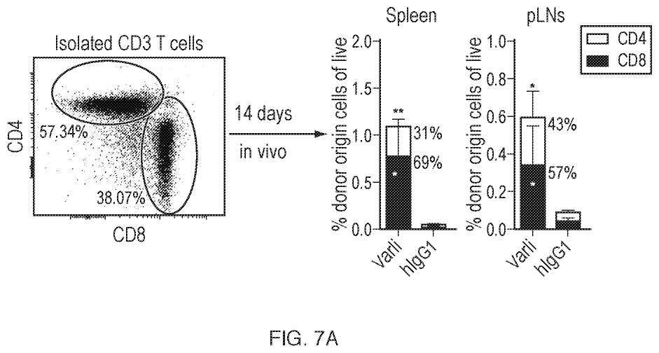

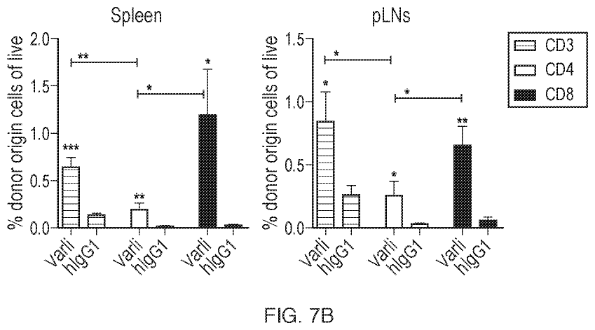

CD3 T-cells were isolated from w.t. mice and the proportions of CD4 and CD8 T-cells were shown in the dot plot (FIG. 7A). These CD3 T cells were labeled with CFSE and transfused at 2×10⁶ per mouse into hCD27^{+/+}mCD27^{-/-} recipients pretreated with 300 μg of varlilumab or hIgG1 on days −14 and −2. Stacked bar graphs show the percentages of donor origin cells out of total live cells in spleens and pLNs with relative percentages of CD8 and CD4 T-cells labeled beside the corresponding bars after 14 days' in vivo expansion (FIG. 7A). CD3, CD4, or CD8 T-cells were isolated from w.t. mice, labeled with CFSE and transfused at 3×10⁶ per mouse into hCD27^{+/+}mCD27^{-/-} recipients pretreated with 200 μg of varlilumab or hIgG1 on days −14 and −2 (FIG. 7B). Data are displayed as percentages of donor origin cells out of total live cells in the spleen and pLNs after 14 days' in vivo expansion.

Data are from one representative experiment of two performed, 3 or 4 mice per group in each study, *p<0.05, **p<0.01, ***p<0.001, indicating statistical significance compared to isotype control or between groups as specified by the horizontal line. The results show that varlilumab pretreatment leads to more CD8 T-cells expansion upon same number or even smaller number of cells transfused relative to CD4 T-cells.

### Example 8: Expansion of Transferred CD8 T-Cells is Abrogated or Reduced Upon Loss of CD27 Signaling

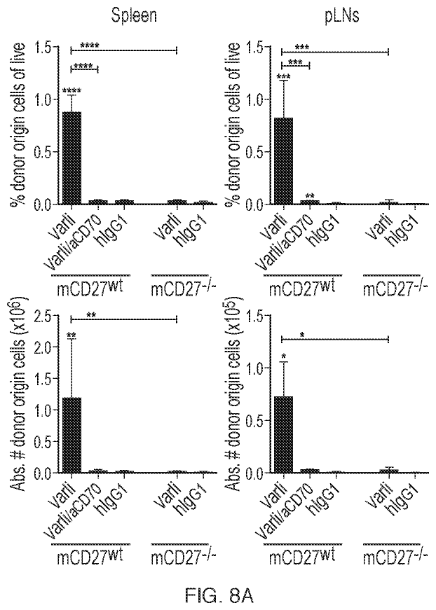

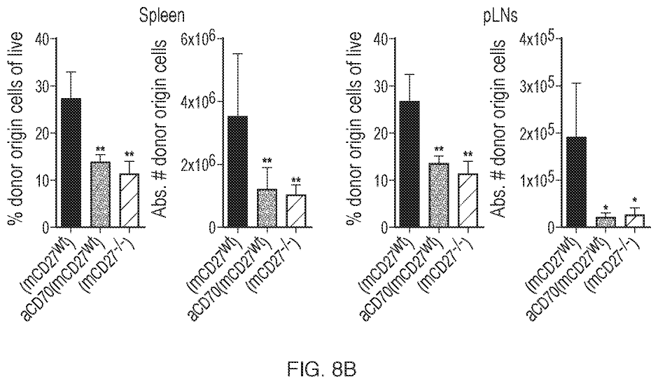

hCD27^{+/+}mCD27^{-/-} mice were injected with 300 μg of varlilumab or hIgG1 on day −14 and −2 plus or minus 200 μg of CD70 blockade on day −2, 1, 4, 7 and 10. These mice were transfused on day 0 with 2×10⁶ CD8 T-cells isolated from w.t. mice (mCD27^{wt}) or CD27 knockout mice (mCD27^{-/-}) as labeled in x-axis (FIG. 8A). Rag2^{-/-} mice (purchased from Taconic) were pretreated with or without the same doses of CD70 blockade and transfused with 2×10⁶ CD8 T-cells isolated from mCD27^{wt} or mCD27^{-/-} donors (FIG. 8B). Spleen and pLNs were harvested on day 14 and processed for flow cytometry analysis. Results are displayed as the percentage of donor origin CD8 T-cells out of total live cells and their absolute numbers in spleen and pLNs (FIGS. 8A and 8B).

Data are from one representative experiment of two performed (n=3 mice per group), *p<0.05, **p<0.01, ***p<0.001, ****p<0.0001. In FIG. 8A, notations above bars indicate statistical significance compared to hIgG1, and horizontal lines indicate statistical significance between the groups specified. In FIG. 8B, notations above bars indicate statistical significance compared to w.t. donor cells without CD70 blockade. The results indicate that the expansion of donor CD8 T-cells is abrogated in varlilumab-pretreated hCD27^{+/+}mCD27^{-/-} recipients (FIG. 8A) and significantly reduced in Rag2^{-/-} recipients (FIG. 8B) upon loss of CD27 signaling through blocking CD70 or using mCD27^{-/-} donor cells.

### Example 9: Expansion of Transferred CD8 T-Cells is Enhanced Upon Adding CD27 Signaling to CD27-Deficient Donor Cells

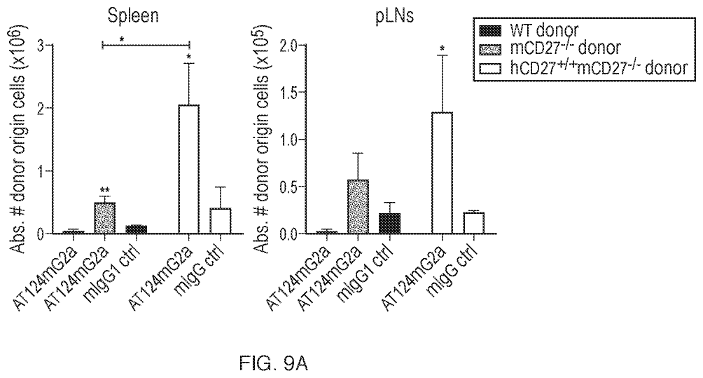

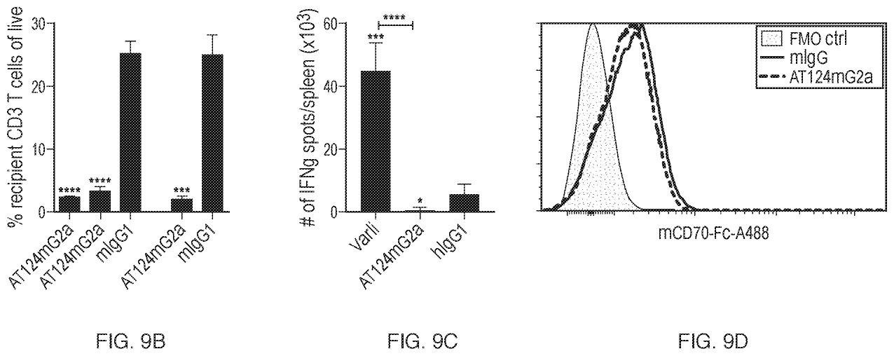

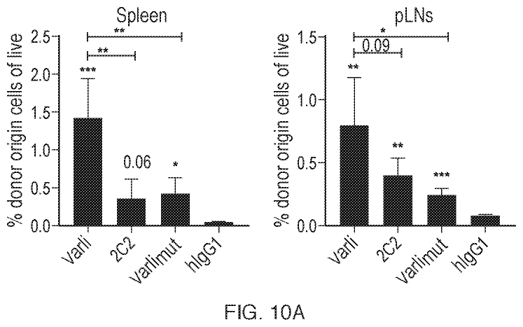

W.t. mice were injected with 50 μg of AT124mG2a or mIgG control on day −7 and −2. On day 0, CD8 T cells isolated from w.t., mCD27^{-.-} or hCD27^{+/+}mCD27^{-/-} mice were labeled with CFSE and transfused at 2×10⁶ per mouse into pretreated recipients that expressed corresponding CD45 congenic markers allowing the discrimination of donor and recipient cells. Shown are the absolute numbers of donor origin cells in the spleen and pLNs after 14 days of in vivo expansion, n=3 mice per group (FIG. 9A), and the percentage of CD3 T cells in recipients' blood on day 0 before cell transfusion (FIG. 9B). Data are from one representative experiment of two performed, *p<0.05, **p<0.01, ***p<0.001, ****p<0.0001 indicating statistical significance compared to isotype control or between groups as specified by the horizontal line.

hCD27^{+/-}mCD27^{wt} mice were injected with 5 mg OVA and 50 μg of varlilumab, AT124mG2a or hIgG1 control on day 0 and spleens were collected 7 days later for ELISPOT analysis. Shown are SIINFEKL-specific IFNγ spots number per spleen, n=4 or 5 mice per group (FIG. 9C). Data are from one representative experiment of two performed, *p<0.05, ***p<0.001, ****p<0.0001, indicating statistical significance compared to isotype control or between groups as specified by the horizontal line.

Splenocytes from w.t. mice were incubated with 10 μg AT124mG2a or mIgG control on ice for 30 minutes and then stained with a fluorescence-labeled mouse CD70-Fc fusion protein (0.5 μg) and Abs for cell surface markers. Shown is a histogram of mCD70-Fc staining on gated CD4⁺CD25⁺ T_{reg} cells (FIG. 9D). No difference is seen in CD70 binding on cells preincubated with AT124mG2a (broken line) or mIgG (gray line). FMO stands for fluorescence minus one, reflecting background fluorescence on the same gated cells.

These results demonstrate that: 1) AT124mG2a induces strong T-cell depletion but neither costimulation nor ligand blocking activities; 2) the residual AT124mG2a that is still present from pretreatment can depleted transfused w.t. CD8 T-cells, leading to a further smaller numbers of donor origin cells relative to isotype control; 3) donor CD8 T cells isolated from hCD27^{+/+}mCD27^{-/-} mice had greater expansion compared to mCD27^{-/-} donor cells in AT124mG2a-pretreated recipients, verifying the role of CD27 signaling.

### Example 10: Both Depleting and Ligand Blocking Activities of Varlilumab Contribute to the Enhanced Expansion of Adoptively Transferred CD8 T Cells

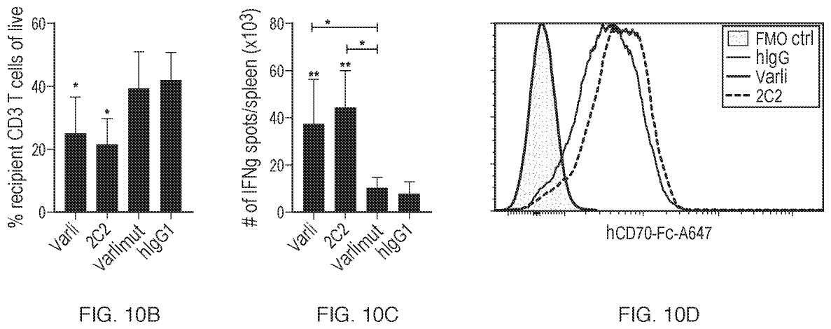

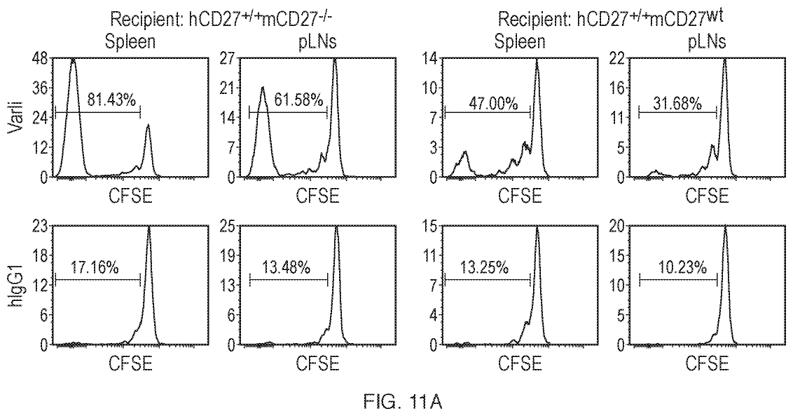

hCD27^{+/+}mCD27^{-/-} mice were injected with 300 μg of varlilumab, 2C2 (a different clone of hCD27 Ab), varliₘᵤₜ (a FC_{D265A} mutated mouse IgG1 isotype of varlilumab) or hIgG1 on days −14 and −2. These mice were bled and then transfused with 2×10⁶ CD8 T-cells on day 0. Shown are the percentages of donor origin cells out of total live cells in the spleen and pLNs after 14 days of in vivo expansion (FIG. 10A) and the percentages of CD3 T cells in recipients' blood on day 0 before transfusion (FIG. 10B). Data are from one representative experiment of two performed, n=5 mice per group, *p<0.05, **p<0.01, ***p<0.001 indicating statistical significance compared to isotype control or between groups as specified by the horizontal line.

hCD27^{+/-}mCD27^{wt} mice were injected with 5 mg OVA and 50 μg of varlilumab, 2C2, varliₘᵤₜ, or hIgG1 on day 0. Spleens were collected 7 days later for ELISPOT analysis. Shown are SIINFEKL-specific IFNγ spots number per spleen, n=5 mice per group (FIG. 10C). Data are from one representative experiment of two performed, *p<0.05, **p<0.01, indicating statistical significance compared to isotype control or between groups as specified by the horizontal line.

Splenocytes from hCD27^{+/+}mCD27^{-/-} mice were incubated with 10 μg Ab as indicated for 30 minutes on ice and then stained with a fluorescently labeled human CD70-Fc fusion protein (0.5 μg) and Abs for cell surface markers. Shown is a histogram of hCD70-Fc staining on gated CD4⁺CD25⁺ T_{reg} cells. While no difference in CD70 binding was seen on cells between preincubated with 2C2 (broken line) and hIgG1 isotype control (gray line), there was no hCD70 binding detected on cells preincubated with varlilumab (black line) (FIG. 10D).

These results demonstrate that pretreatment with varlilumab (possessing T cell depletion and ligand blocking activities) leads to greater expansion of transfused CD8 T-cells compared to 2C2 that has comparable depleting and agonistic activities with varlilumab but no ligand blocking activity or varlimut that blocks ligand binding but has no depleting and agonistic activities. Therefore, T cell depleting and ligand blocking activities, perhaps also agonistic activity, working together achieve the optimal transferred cell expansion.

### Example 11: Donor Cell Expansion is Decreased in Recipients That Have Competent CD27 Signaling

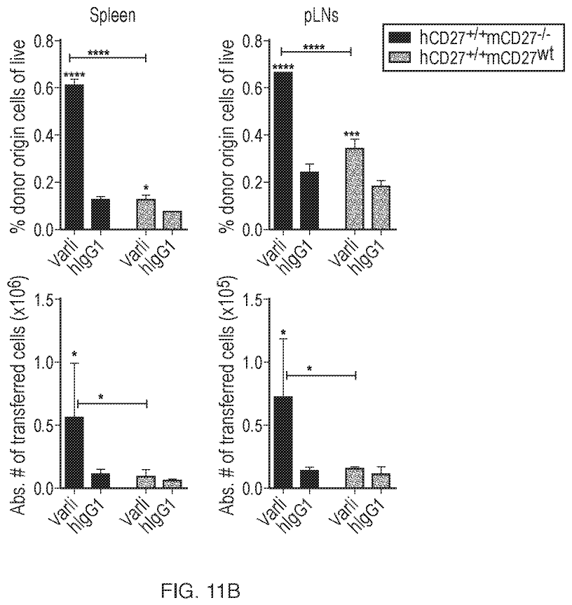

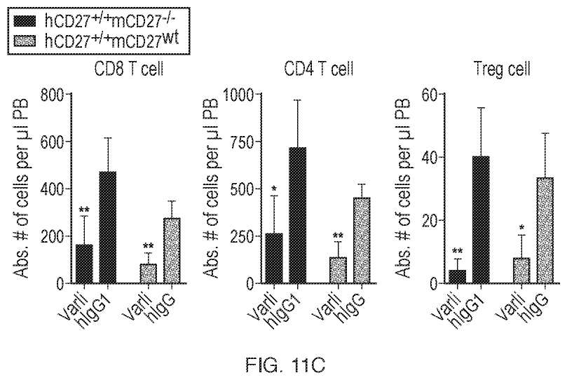

hCD27^{+/+}mCD27^{-/-} or hCD27^{+/+}mCD27^{wt} mice were injected with 200 μg of varlilumab or hIgG1 on days −7 and −2 and transfused with 3×10⁶ CFSE-labeled w.t. CD8 T cells on day 0. Spleens and pLNs were collected on day 14 and processed for flow cytometry analysis. Representative flow cytometry histograms of CFSE dilution were depicted in FIG. 11A, and the percentage of donor origin CD8 T-cells out of total live cells and their absolute numbers in spleen and pLNs were depicted in FIG. 11B (n=3 mice per group). hCD27^{+/+}mCD27^{-/-} or hCD27^{+/+}mCD27^{wt} mice were injected with 300 μg of varlilumab or hIgG1 on day 0 and blood was collected 14 days later for flow cytometry analysis. CD8, CD4 and T_{reg} cell counts per μl blood were depicted in FIG. 11C (n=6 mice per group).

Data are from one representative experiment of two performed, *p<0.05, **p<0.01, ***p<0.001, ****p<0.0001, indicating statistical significance compared to isotype within same strain of recipients or between the two strains of recipient mice as specified by the horizontal line. Results show the remarkable transferred cell expansion in hCD27^{+/+}mCD27^{-/-} recipients lacking CD27 signaling (hCD27 was blocked by the injected varlilumab and mCD27 was knocked out) and a moderate expansion in recipients having competent CD27 signaling (hCD27^{+/+}mCD27^{wt}) following the same varlilumab pretreatment that led to the same extent of T cell reduction in these two strains of recipients.

### Example 12: Proliferation of Endogenous Cells is Decreased in Mice Lacking of CD27 Signaling After Varlilumab Injection

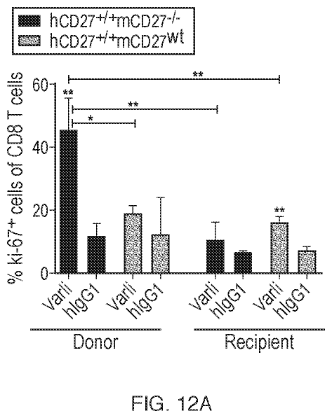

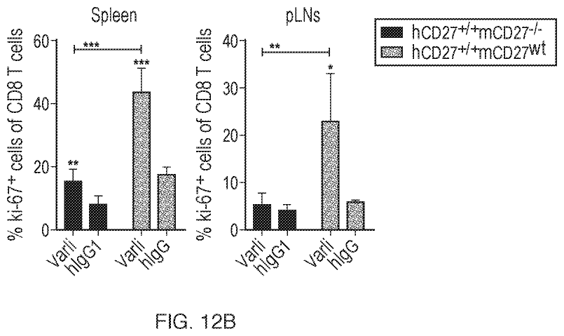

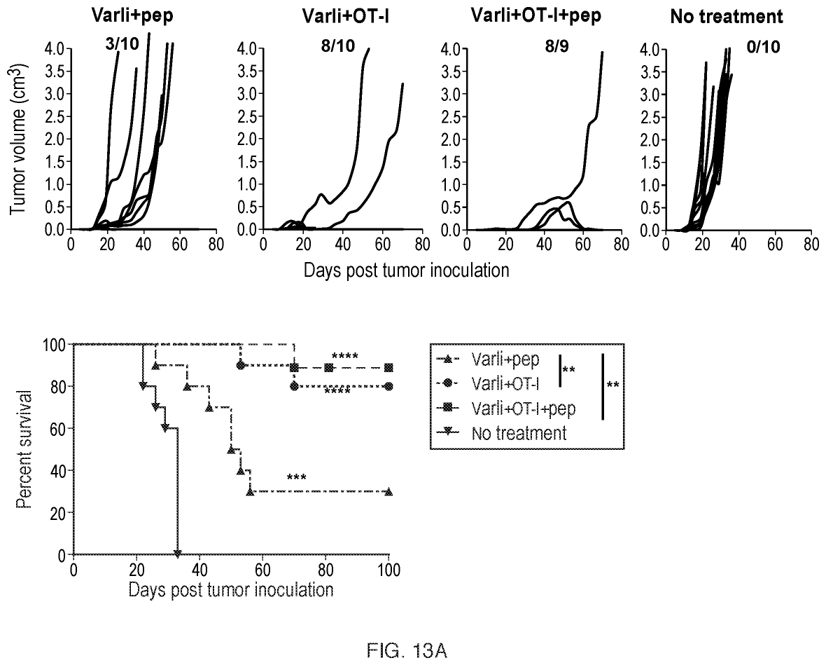

Percentage of ki-67⁺ in gated donor origin or recipient origin CD8 T cells in the spleen of the same mice as in FIGS. 11A and 11B were depicted in FIG. 12A. Percentages of Ki-67⁺ cells in gated CD8 T cells in the spleen and pLNs of the same mice as in FIG. 11C were depicted in FIG. 12B.

Data are from one representative experiment of two performed, 3-5 mice per group in each study, *p<0.05, **p<0.01, ***p<0.001, indicating statistical significance compared to isotype control within same strain of recipients or between the two strains of recipient mice or between donor and recipient cells as specified by the horizontal line. Results show that CD8 T-cell proliferation is significantly higher in donor origin cells than that in recipient origin cells in hCD27^{+/+}mCD27^{-/-} mice while that has no difference in hCD27^{+/+}mCD27^{wt} mice following the same varlilumab pretreatment (FIG. 12A). In the comparison of Ki-67⁺ in endogenous CD8 T cells following varlilumab treatment without donor cell transfer, more proliferation is observed in hCD27^{+/+}mCD27^{wt} mice than that in hCD27^{+/+}mCD27^{-/-} mice (FIG. 12B). Collectively, it is suggested that depriving recipient endogenous cells from competing the limiting CD27 ligand leads to decreased proliferation in recipient cells and increased proliferation in donor cells in hCD27^{+/+}mCD27^{-/-} mice.

### Example 13: Pretreatment with Varlilumab Enhances Antitumor Efficacy of Adoptive T-Cell Therapy

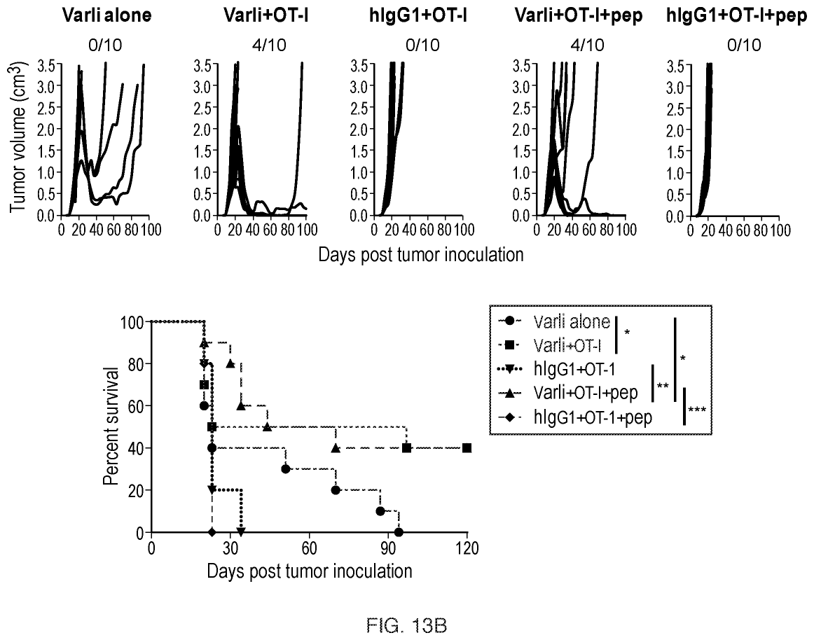

hCD27^{+/+}mCD27^{-/-} mice were inoculated subcutaneously (s.c.) with 0.5×10⁶ E.G7 cells on day 0. These mice were injected with 300 μg of varlilumab 2 days before and 5 days after tumor inoculation (day −2 and 5), 2×10⁶ OT-I T-cells i.v. on day 7, and 100 μg of SIINFEKL peptide i.p. on day 8 (FIG. 13A), or given delayed therapy, i.e., same dose of varlilumab or hIgG1 on day 6 and 14, same number of OT-I T-cells on day 16, and 20 μg of SIINFEKL peptide on day 17 (FIG. 13B). Tumor volumes were measured twice a week and calculated using a modified ellipsoid formula [V=½ (length×width²)], n=10 mice per group. Tumor growth curves are depicted with inserted fraction for survived mice out of total mice per treatment. Kaplan-Meier survival plots are depicted, *p<0.05, **p<0.01, ***p<0.001, ****<0.0001, indicating statistical significance compared to no treatment or between the groups as specified by the vertical lines. The results reveal that varlilumab pretreatment followed by OT-I T-cell adoptively transfer has stronger antitumor efficacy than varlilumab alone, and varlilumab pretreatment enhances antitumor efficacy of OT-I T-cells compared to hIgG1 isotype control. There is no difference in long-term survival between with or without SIINFEKL peptide injection following OT-I T cell transfer.

### Example 14: Compare Recipient Cell Depletion and Donor Cell Expansion Between Conditioning Treatment with Varlilumab Versus Current Regimen with Cy and Flu

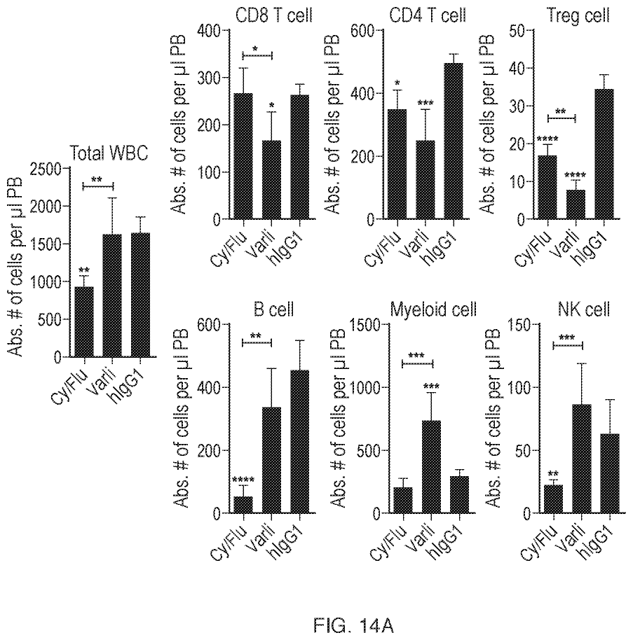

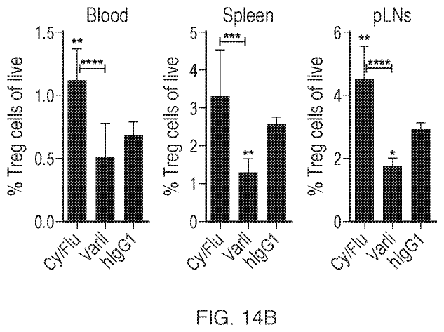

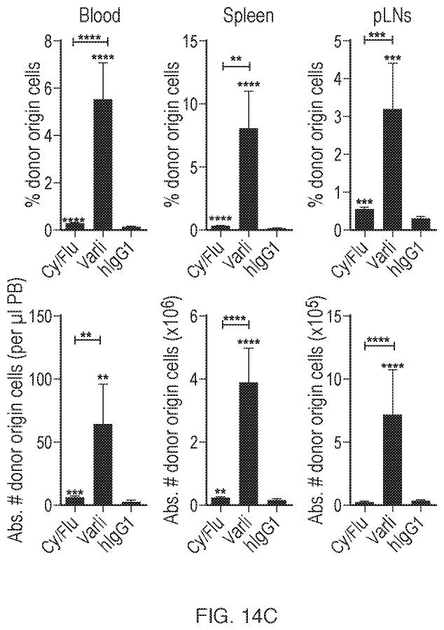

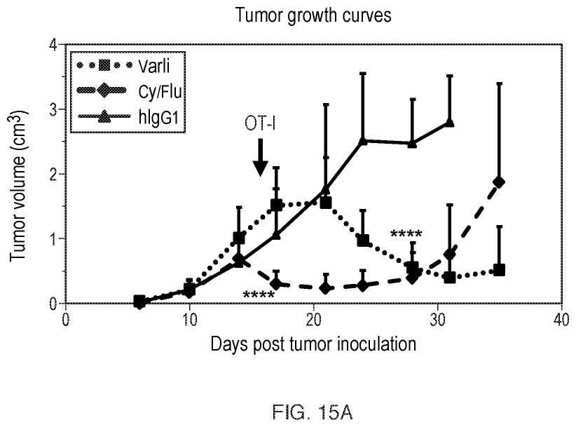

hCD27^{+/+}mCD27^{-/-} mice were injected with 300 μg of varlilumab on day −14 and −2 or Cy 1 mg and Flu 0.1 mg on day −4, −3 and −2. One group of same mice injected with hIgG1 serves as control. All these mice were bled on day 0 and then transfused with 2×10⁶ w.t. CD8 T-cells on the same day. PB, spleens, and pLNs were collected 14 days later and processed for flow cytometry analysis. FIG. 14A shows total and differential counts of WBC per μl blood on the day of adoptive transfer (day 0), revealing that chemotherapeutic drug Cy and Flu pretreatment reduces all subtypes of WBC, especially B-cells, and leads to a decrease in total WBC, while varlilumab only depletes T-cells with the most reduction in T_{reg} population and does not attack myeloid cells, B-cells and NK cells. FIG. 14B shows the percentage of T_{reg} cells out of total live cells in blood, spleen and pLNs on day 14, revealing that T_{reg} remains low in varlilumab pretreated mice on day 14, while it is fully recovered or rebound in mice pretreated with Cy and Flu. FIG. 14C shows the percentage of donor origin CD8 T-cells out of total live cells and their absolute number in blood, spleen and pLNs, revealing that donor cell expansion is significantly greater in Cy and Flu pretreated recipients versus control mice, and further increased in varlilumab pretreated mice.

Data are from one representative experiment of two performed, n=5 mice per group, *p<0.05, **p<0.01, ***p<0.001, ****p<0.0001, indicating statistical significance compared to control group or between the groups specified by the horizontal lines. The results demonstrate that varlilumab conditioning treatment is superior to Cy and Flu combo, in term of T-cell-restricted depletion and much stronger enhancement on the expansion of adoptively transferred T-cells.

### Example 15: Varlilumab is Superior to Cy and Flu as Conditioning Treatment for Adoptive T Cell Antitumor Activity

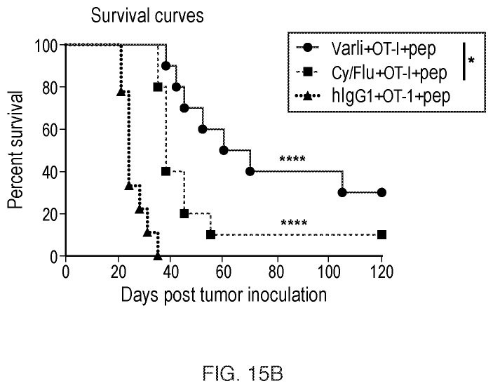

hCD27^{+/+}mCD27^{-/-} mice were inoculated s.c. with 0.5×10⁶ E.G7 cells on day 0. These mice were injected i.p. with 300 μg of varlilumab or hIgG1 on day 7 and 14 or 1 mg of Cy and 0.1 mg of Flu on day 13 and 14, 2×10⁶ OT-I T-cells i.v. on day 16, and 20 μg of SIINFEKL peptide i.p. on day 17. Tumor volumes were measured twice a week and calculated using a modified ellipsoid formula [V=½ (length×width²)]. Tumor growth curves (mean±SD) and Kaplan-Meier survival plots are depicted (FIGS. 15A and 15B). Data are from one representative experiment of two performed (n=10 mice per group), *p<0.05, ****p<0.0001, indicating statistical significance compared to hIgG1 control or between the groups specified by the vertical line. The results demonstrate that varlilumab or the chemotherapy regimens as conditioning treatment facilitates OT-I T-cells antitumor activity compared to hIgG1 control, and the enhancement of varlilumab pretreatment is further greater than Cy and Flu regimen.

### Example 16: Varlilumab and Current Conditioning Regimen Synergistically Enhance OT-I T-Cells Antitumor Activity

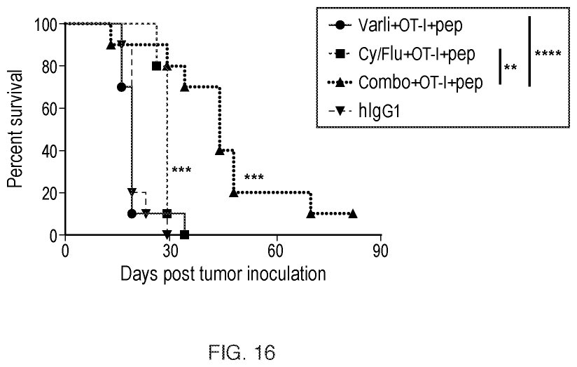

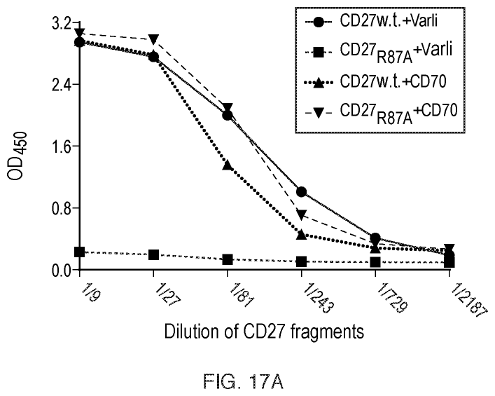

hCD27^{+/+}mCD27^{-/-} mice were inoculated s.c. with 0.5×10⁶ E.G7 cells on day 0, and treated with 300 μg of varlilumab or hIgG1 on day 14 and/or 1 mg of Cy and 0.1 mg of Flu on day 13 and 14. 2×10⁶ OT-I T-cells were injected i.v. on day 16, and 20 μg of SIINFEKL peptide i.p. on day 17. Tumor volumes were measured twice a week and calculated using a modified ellipsoid formula [V=½ (length×width²)]. Kaplan-Meier survival plots are depicted in FIG. 16, n=10 mice per group, **p<0.01, ***p<0.001, ****p<0.0001, indicating statistical significance compared to hIgG1 control or between the groups specified by the vertical line. The results demonstrate that one delayed dose of varlilumab, even though no conditioning effect by itself, synergizes Cy and Flu for OT-I T-cells antitumor activity.

### Example 17: CD27_{R87A} Single Mutation Abolishes Varlilumab Recognition but Retains CD70 Binding

CD27 extracellular domain spanning amino acid residue 1-110 w.t. sequence and R87A mutation sequence according to Kabat numbering were cloned into an expression vector fused with a human kappa chain in the N-terminus and a flag-tag in the C-terminus. The CD27 fragments were expressed by transient transfection and quantified by an ELISA measuring concentration of human kappa chain in the culture supernatant.

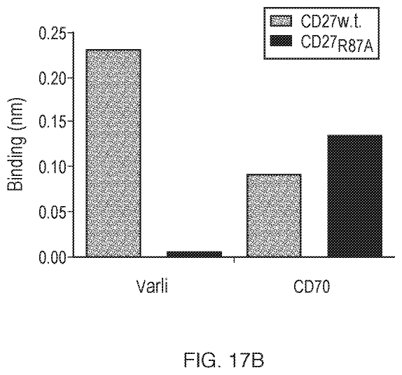

ELISA. CD27 fragment-containing supernatants were serially diluted and captured by microplate-bound anti-flag Ab, incubated with varlilumab or CD70-biotin and then detected with HRP-conjugated secondary Ab or streptavidin and subtract. Shown are readouts of OD₄₅₀ against dilutions of the w.t. and mutant CD27 fragments upon varlilumab or CD70 binding, illustrating that varlilumab recognizes w.t. CD27 but not CD27_{R87A}, while CD70 binds both (FIG. 17A).

Fortebio Octet. Anti-human Fc biosensors were loaded with either varlilumab, rhCD70-Fc or buffer only, and then exposed to CD27 fragment-containing supernatants. Binding was reported as nanometer (nm) shift at the end of the association step after subtraction of non-specific background. FIG. 17B shows the lost binding affinity of varlilumab to CD27_{R87A} but not to CD70.

SUMMARY OF SEQUENCE LISTING

| | |
|---|---|
| SEQ ID NO: 1 | MARPHPWWLC VLGTLVGLSA TPAPKSCPER HYWAQGKLCC |
| Human CD27 | QMCEPGTFLV KDCDQHRKAA QCDPCIPGVS FSPDHHTRPH |
| | CESCRHCNSG LLVRNCTITA NAECACRNGW QCRDKECTEC |
| | DPLPNPSLTA RSSQALSPHP QPTHLPYVSE MLEARTAGHM |
| | QTLADFRQLP ARTLSTHWPP QRSLCSSDFI RILVIFSGMF |
| | LVFTLAGALF LHQRRKYRSN KGESPVEPAE PCRYSCPREE |
| | EGSTIPIQED YRKPEPACSP |
| SEQ ID NO: 2 | MPEEGSGCSV RRRPYGCVLR AALVPLVAGL VICLVVCIQR |
| Human CD70 | FAQAQQQLPL ESLGWDVAEL QLNHTGPQQD PRLYWQGGPA |
| | LGRSFLHGPE LDKGQLRIHR DGIYMVHIQV TLAICSSTTA |
| | SRHHPTTLAV GICSPASRSI SLLRLSFHQG CTIASQRLTP |
| | LARGDTLCTN LTGTLLPSRN TDETFFGVQW VRP |
| SEQ ID NO: 3 | QVQLVESGGGVVQPGRSLRLSCAASGFTFS |
| 1F5 VH amino | SYDMHWVRQAPGKGLEWVAVIWYDGSNKYY |
| acid sequence | ADSVKGRFTISRDNSKNTLYLQMNSLRAED |
| | TAVYYCARGSGNWGFFDYWGQGTLVTVSS |
| SEQ ID NO: 4 | DIQMTQSPSSLSASVGDRVTITCRASQGIS |
| 1F5 VL amino | RWLAWYQQKPEKAPKSLIYAASSLQSGVPS |
| acid sequence | RFSGSGSGTDFTLTISSLQPEDFATYYCQQ |
| | YNTYPRTFGQGTKVEIK |
| SEQ ID NO: 5 | GFTFSSYD |
| 1F5 VH CDR1 | |
| amino acid | |
| sequence | |
| SEQ ID NO: 6 | IWYDGSNK |
| 1F5 VH CDR2 | |
| amino acid | |
| sequence | |
| SEQ ID NO: 7 | ARGSGNWGFFDY |
| 1F5 VH CDR3 | |
| amino acid | |
| sequence | |
| SEQ ID NO: 8 | QGISRW |
| 1F5 VL CDR1 | |
| amino acid | |
| sequence | |
| SEQ ID NO: 9 | AAS |
| 1F5 VL CDR2 | |
| amino acid | |
| sequence | |
| SEQ ID NO: 10 | QQYNTYPRT |
| 1F5 VL CDR3 | |
| amino add | |
| sequence | |
| SEQ ID NO: 11 | SIINFEKL |
| Ovalbumin | |
| peptide | |
| SEQ ID NO: 12 | QVQLVESGGG VVQPGRSLRL SCAASGFTFS SYDIHWVRQA |
| 2C2 VH amino | PGKGLEWVAV IWNDGSNKYY ADSVKGRFTI SRDNSTNSLF |
| acid sequence | LQMNSLRAED TAVYYCVGGT ADLEHWDQGT LVTVSS |
| SEQ ID NO: 13 | DIQMTQSPSS LSASVGDRVT ITCRASQGIS SWLAWYQQKP |
| 2C2 VL amino | EKAPKSLIYA ASSLQSGVPS RFSGSGSGTD FTLTISSLQP |
| aacd sequence | EDFATYYCQQ YNSYPLTFGG GTKVEIK |
| SEQ ID NO: 14 | QVQLVESGGG VVQPGRSLRL SCATSGFTFS SYDMHWVRQA |
| 3H12 VH amino | PGKGLEWVAV IWYDGSNKYY ADSVKGRFTI SRDNSKNTLY |
| acid sequence | LQMNSLGDED TAVYYCARGS GNWGFFDYWG QGTLVTVSS |
| SEQ ID NO: 15 | DIQMTQSPSS LSASVGDRVT ITCRASQGIS RWLAWYQQKP |
| 3H12 VL amino | EKAPKSLIYA ASSLQSGVPS RFSGSGSGTD FTLTISSLQP |
| acid sequence | EDFATYYCQQ YNTYPRTFGQ GTKVEIK |
| SEQ ID NO: 16 | QVQLVESGGG VVQPGRSLRL SCAASGFTLS SHDIHWVRQA |
| 2G9 VH amino | PGKGLEWVAV IWNDGSNKYY ADSVKGRFTI SRDNSTNSLF |
| acid sequence | LQMNSLRAED TAVYYCVRGT ADLEHWDQGT LVTVSS |
| SEQ ID NO: 17 | DIQMTQSPSS LSASVGDRVT ITCRASQGIS SWLAWYQQKP |
| 2G9 VL amino | EKAPKSLIYA ASSLQSGVPS RFSGSGSGTD FTLTISSLQP |
| acid sequence | EDFATYYCQQ YNSYPLTFGG GTKVEIK |
| SEQ ID NO: 18 | QVQLVESGGG VVQPGRSLRL SCAASGFTFN IYDMHWVRQA |
| 1H8 VH amino | PGKGLEWVAV IWYDGSNQYY ADSVKGRFTI SRDNSKNTLY |
| acid sequence | LQMNILRAED TAVYYCARGT HWGYFDYWGQ GTLVTVSS |
| SEQ ID NO: 19 | DIQMTQSPSS LSASVGDRVT ITCRASQGIS SWLAWYQQKP |
| 1H8 VL amino | EKAPKSLIYA ASNLQSGVPS RFSGSGSGTD FTLTISSLQP |
| acid sequence | EDFATYYCQQ YNSYPRTFGQ GTKVEIK |
| SEQ ID NO: 20 | QVQLVESGGG VVQPGRSLRL SCAASGFTFS HYGMHWVRQA |
| 3A10 VH amino | PGKGPEWVAI IWYDGSNKYY ADSVKGRFTI SRDNSKNTLD |
| acid sequence | LQMNSLRAED TAVYYCARDG WTTMVRGLNV FDIWGQGTMV |
| | TVSS |
| SEQ ID NO: 21 | DIQMTQSPSS LSASVGDRVT ITCRASQDIS SWLAWYQQKP |
| 3A10 VL amino | EKAPKSLIYA ASSLQSGVPS RFSGSGSGTD FTLTISSLQP |
| acid sequence | EDFATYYCQQ YNSYPPTFGQ GTRLEIK |
| SEQ ID NO: 22 | EVQLVESGGG LVQPGGSLRL SCAASGFTFS SYWMAWVRQA |
| 3H8 VH amino | PGKGLEWLGN IKQDGSEKYY VDSVKGRFTI SRDNAKNSLY |
| acid sequence | LQMNSLRAED TAVYYCVREL GMDWYFDLWG RGTLVTVSS |
| SEQ ID NO: 23 | EIVLTQSPAT LSLSPGERAT LSCRASQSVD SYLAWYQQKP |
| 3H8 VL amino | GQAPRLLIYD ASNRATGIPA RFSGSGSGTD FTLTISNLEP |
| acid sequence | EDFAVYYCQQ RSNWPPTFGQ GTKVEIK |
| SEQ ID NO: 24 | QVQLVESGGG VVQPGRSLRL SCAASGFSFS SYGMHWVRQA |
| 1G5 VH amino | PGKGLEWVAL LWYDGSHKDF ADSVKGRFTI SRDNSKNTLD |
| acid sequence | LQMNSLRAED TAVYYCAREG LAVPGHWYFD LWGRGTLVTV |
| | SS |
| SEQ ID NO: 25 | AIQLTQSPSS LSASVGDRVT ITCRASQGIS SALAWYQQKP |
| 1G5 VL amino | GKAPKLLIYD ASSLESGVPS RFSGSGSGTD FTLTISSLQP |
| acid sequence | EDFATYYCQQ FNTYPRTFGQ GTKVEIK |
| SEQ ID NO: 26 | GFTFSSYD |
| 2C2 VH CDR1 | |
| amino acid | |
| sequence | |
| SEQ ID NO: 27 | IWNDGSNK |
| 2C2 VH CDR2 | |
| amino acid | |
| sequence | |
| SEQ ID NO: 28 | VGGTADLEHWDQ |
| 2C2 VH CDR3 | |
| amino acid | |
| sequence | |
| SEQ ID NO: 29 | QGISSW |
| 2C2 VL CDR1 | |
| amino acid | |
| sequence | |
| SEQ ID NO: 30 | AAS |
| 2C2 VL CDR2 | |
| amino acid | |
| sequence | |
| SEQ ID NO: 31 | QQYNSYPLT |
| 2C2 VL CDR3 | |
| amino acid | |
| sequence | |
| SEQ ID NO: 32 | GFTFSSYD |
| 3H12 VH CDR1 | |
| amino acid | |
| sequence | |
| SEQ ID NO: 33 | IWYDGSNK |
| 3H12 VH CDR2 | |
| amino acid | |
| sequence | |
| SEQ ID NO: 34 | ARGSGNWGFFDY |
| 3H12 VH CDR3 | |
| amino acid | |
| sequence | |
| SEQ ID NO: 35 | QGISRW |
| 3H12 VL CDR1 | |
| amino acid | |
| sequence | |
| SEQ ID NO: 36 | AAS |
| 3H12 VL CDR2 | |
| amino acid | |
| sequence | |
| SEQ ID NO: 37 | QQYNTYPRT |
| 3H12 VL CDR3 | |
| amino acid | |
| sequence | |
| SEQ ID NO: 38 | GFTLSSHD |
| 2G9 VH CDR1 | |
| amino acid | |
| sequence | |
| SEQ ID NO: 39 | IWNDGSNK |
| 2G9 VH CDR2 | |
| amino acid | |
| sequence | |
| SEQ ID NO: 40 | VRGTADLEHWDQ |
| 2G9 VH CDR3 | |
| amino acid | |
| sequence | |
| SEQ ID NO: 41 | QGISSW |
| 2G9 VL CDR1 | |
| amino acid | |
| sequence | |
| SEQ ID NO: 42 | AAS |
| 2G9 VL CDR2 | |
| amino acid | |
| sequence | |
| SEQ ID NO: 43 | QQYNSYPLT |
| 2G9 VL CDR3 | |
| amino acid | |
| sequence | |
| SEQ ID NO: 44 | GFTFNIYD |
| 1H8 VH CDR1 | |
| amino acid | |
| sequence | |
| SEQ ID NO: 45 | IWYDGSNQ |
| 1H8 VH CDR2 | |
| amino acid | |
| sequence | |
| SEQ ID NO: 46 | ARGTHWGYFDY |
| 1H8 VH CDR3 | |
| amino acid | |
| sequence | |
| SEQ ID NO: 47 | QGISSW |
| 1H8 VL CDR1 | |
| amino acid | |
| sequence | |
| SEQ ID NO: 48 | AAS |
| 1H8 VL CDR2 | |
| amino acid | |
| sequence | |
| SEQ ID NO: 49 | QQYNSYPRT |
| 1H8 VL CDR3 | |
| amino acid | |
| sequence | |
| SEQ ID NO: 50 | GFTFSHYG |
| 3A10 VH CDR1 | |
| amino acid | |
| sequence | |
| SEQ ID NO: 51 | IWYDGSNK |
| 3A10 VH CDR2 | |
| amino acid | |
| sequence | |
| SEQ ID NO: 52 | ARDGWTTMVRGLNVFDI |
| 3A10 VH CDR3 | |
| amino acid | |
| sequence | |
| SEQ ID NO: 53 | QDISSW |
| 3A10 VL CDR1 | |
| amino acid | |
| sequence | |
| SEQ ID NO: 54 | AAS |
| 3A10 VL CDR2 | |
| amino acid | |
| sequence | |
| SEQ ID NO: 55 | QQYNSYPPT |
| 3A10 VL CDR3 | |
| amino acid | |
| sequence | |
| SEQ ID NO: 56 | GFTFSSYW |
| 3H8 VH CDR1 | |
| amino acid | |
| sequence | |
| SEQ ID NO: 57 | IKQDGSEK |
| 3H8 VH CDR2 | |
| amino acid | |
| sequence | |
| SEQ ID NO: 58 | VRELGMDWYFDL |
| 3H8 VH CDR3 | |
| amino acid | |
| sequence | |
| SEQ ID NO: 59 | QSVDSY |
| 3H8 VL CDR1 | |
| amino acid | |
| sequence | |
| SEQ ID NO: 60 | DAS |
| 3H8 VL CDR2 | |
| amino acid | |
| sequence | |
| SEQ ID NO: 61 | QQRSNWPPT |
| 3H8 VL CDR3 | |
| amino acid | |
| sequence | |
| SEQ ID NO: 62 | GFSFSSYG |
| 1G5 VH CDR1 | |
| amino acid | |
| sequence | |
| SEQ ID NO: 63 | LLWYDGSHK |
| 1G5 VH CDR2 | |
| amino acid | |
| sequence | |
| SEQ ID NO: 64 | AREGLAVPGHWYFDL |
| 1G5 VH CDR3 | |
| amino acid | |
| sequence | |
| SEQ ID NO: 65 | QGISSA |
| 1G5 VL CDR1 | |
| amino acid | |
| sequence | |
| SEQ ID NO: 66 | DAS |
| 1G5 VL CDR2 | |
| amino acid | |
| sequence | |
| SEQ ID NO: 67 | QQFNTYPRT |
| 1G5 VL CDR3 | |
| amino acid | |
| sequence | |
| SEQ ID NO: 68 | QVQLVESGGG VVQPGRSLRL SCAASGFTFS SYDMHWVRQA |
| Varlilumab Heavy | PGKGLEWVAV IWYDGSNKYY ADSVKGRFTI SRDNSKNTLY |
| Chain | LQMNSLRAED TAVYYCARGS GNWGFFDYWG QGTLVTVSSA |
| | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCDKTHTCPP CPAPELLGGP |
| | SVFLFPPKPK DTLMISRTPE VTCVVVDVSH EDPEVKFNWY |
| | VDGVEVHNAK TKPREEQYNS TYRVVSVLTV LHQDWLNGKE |
| | YKCKVSNKAL PAPIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGKG SS |
| SEQ ID NO: 69 | DIQMTQSPSS LSASVGDRVT ITCRASQGIS RWLAWYQQKP |
| Varlilumab Light | EKAPKSLIYA ASSLQSGVPS RFSGSGSGTD FTLTISSLQP |
| Chain | EDFATYYCQQ YNTYPRTFGQ GTKVEIKRTV AAPSVFIFPP |
| | SDEQLKSGTA SVVCLLNNFY PREAKVQWKV DNALQSGNSQ |
| | ESVTEQDSKD STYSLSSTLT LSKADYEKHK VYACEVTHQG |
| | LSSPVTKSFN RGEC |
| SEQ ID NO: 70 | MARPHPWWLC VLGTLVGLSA TPAPKSCPER HYWAQGKLCC |
| CD27_{R87A} | QMCEPGTFLV KDCDQHRKAA QCDPCIPGVS FSPDHHTRPH |
| (Human CD27 | CESCRHCNSG LLVRNCTITA NAECACANGW QCRDKECTEC |
| with leader | DPLPNPSLTA RSSQALSPHP QPTHLPYVSE MLEARTAGHM |
| sequence at | QTLADFRQLP ARTLSTHWPP QRSLCSSDFI RILVIFSGMF |
| amino acids 1-20 | LVFTLAGALF LHQRRKYRSN KGESPVEPAE PCRYSCPREE |
| (underlined) and | EGSTIPIQED YRKPEPACSP |
| including R87A | |
| mutation | |
| (underlined) | |
| SEQ ID NO: 71 | TPAPKSCPER HYWAQGKLCC QMCEPGTFLV KDCDQHRKAA |
| CD27_{R87A} | QCDPCIPGVS FSPDHHTRPH CESCRHCNSG LLVRNCTITA |
| (Human CD27 | NAECACANGW QCRDKECTEC DPLPNPSLTA RSSQALSPHP |
| including R87A | QPTHLPYVSE MLEARTAGHM QTLADFRQLP ARTLSTHWPP |
| mutation | QRSLCSSDFI RILVIFSGMF LVFTLAGALF LHQRRKYRSN |
| (underlined) | KGESPVEPAE PCRYSCPREE EGSTIPIQED YRKPEPACSP |

1. A method of conditioning treatment for adoptive cell therapy (ACT) in a subject in need thereof comprising:
i) administering a CD27 antibody to the subject; and
ii) transferring autogenic or allogenic T-cells to the subject.

2. A method of treating cancer in a subject in need thereof comprising the steps of:
i) administering a CD27 antibody to the subject; and
ii) transferring autogenic or allogenic T-cells to the subject,
thereby treating cancer in the subject.

3. The method of any of the preceding claims wherein the CD27 antibody causes depletion of regulatory T-cells (T_{reg}), CD4-Th and/or CD8 T-cells in the subject.

4. The method of any of the preceding claims wherein the CD27 antibody causes preferential depletion of regulatory T-cells (T_{reg}) compared to CD8 cells in the subject.

5. The method of any of the preceding claims wherein the CD27 antibody causes blocking of ligand CD70 binding with CD27 receptor.

6. The method of any of the preceding claims wherein the transferred T-cells are genetically engineered T-cells.

7. The method of claim 6 wherein the genetically engineered T-cells express a chimeric antigen receptor (CAR) or a T-cell receptor (TCR) which recognizes a tumor-associated antigen.

8. The method of claim 6 or claim 7 wherein the genetically engineered T-cells express a mutated human CD27 receptor such that the transferred T-cells are significantly less depleted by the CD27 antibody than recipient's T-cells and/or the transferred T-cells can respond to CD70 ligation while recipient's CD27 is blocked by the CD27 antibody.

9. The method of claim 8 wherein the human CD27 receptor comprises the mutation R87A as set forth in SEQ ID NO: 71.

10. The method of any of the preceding claims wherein the transferred T-cells are tumor infiltrating lymphocytes (TILs).

11. The method of any of the preceding claims wherein the transferred and expanded T-cells display an effector phenotype and function.

12. The method of any of the preceding claims wherein the transferred and expanded T-cells respond to antigen stimulation.

13. The method of any of the preceding claims wherein the transferred and expanded T-cells display antitumor activity.

14. The method of any of the preceding claims wherein the CD27 antibody is a depleting antibody.

15. The method of any of the preceding claims wherein the CD27 antibody is an IgG1 antibody.

16. The method of any of the preceding claims wherein the CD27 antibody is able to block CD70-CD27 interaction.

17. The method of claim 16, wherein the CD27 antibody comprises CDRH1, CDRH2, and CDRH3 sequences comprising the amino acid sequences set forth in SEQ ID NOs: 5, 6, and 7, respectively, and CDRL1, CDRL2, and CDRL3 sequences comprising the amino acid sequences set forth in SEQ ID NOs: 8, 9, and 10, respectively.

18. The method of claim 17, wherein the CD27 antibody comprises variable heavy and variable light chain amino acid sequences set forth in SEQ ID NO: 3 and SEQ ID NO: 4, respectively.

19. The method of claim 18 wherein the CD27 antibody is varlilumab.

20. The method of claim 2 wherein the cancer is selected from the group consisting of leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma, marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

21. The method of any of the preceding claims wherein the CD27 antibody is administered at least 12 hours before the T-cells are transferred.

22. The method of claim 21 wherein the CD27 antibody is administered at least 24 hours before the T-cells are transferred.

23. The method of claim 22 wherein the CD27 antibody is administered at least 48 hours before the T-cells are transferred.

24. The method of claim 23 wherein the CD27 antibody is administered approximately 7 days before, or approximately 14 days before, and again approximately 2 days before the T-cells are transferred.

25. The method of any of the preceding claims wherein the T-cells are administered by intravenous infusion.

26. Use of a CD27 antibody as a conditioning agent in adoptive T-cell therapy (ACT).

27. A CD27 antibody for use as a conditioning agent in adoptive T-cell therapy (ACT). A CD27 antibody for use as a conditioning agent in adoptive T-cell therapy to replace or combine with Cy and Flu combo.

28. A genetically engineered T-cell which expresses a mutated human CD27 (hCD27) receptor such that the engineered T-cell is activated by recipient endogenous human CD70 but is significantly less depleted by an anti-hCD27 antibody than recipient T-cells.

29. The genetically engineered T-cell of claim 28, wherein the mutated hCD27 receptor comprises a mutation that reduces binding to a CD27 antibody.

30. The genetically engineered T-cell of claim 28, wherein the mutation abolishes binding of varlilumab to mutant hCD27 receptor.

31. The genetically engineered T-cell of claim 28 wherein the mutated hCD27 receptor comprises the mutation R87A as set forth in SEQ ID NO: 71.

32. Use of the genetically engineered T-cell of any one of claims 28-31 in the method of any one of the preceding claims.


SEQUENCE LISTING (71 sequences)

SEQ ID NO: 1 - protein, 260 aa, Homo sapiens
  misc_feature: Human CD27
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 2 - protein, 193 aa, Homo sapiens
  misc_feature: Human CD70
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 3 - protein, 119 aa, Artificial Sequence
  Synthetic 1F5 VH amino acid sequence
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSS

SEQ ID NO: 4 - protein, 107 aa, Artificial Sequence
  Synthetic 1F5 VL amino acid sequence
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 5 - protein, 8 aa, Artificial Sequence
  Synthetic 1F5 VH CDR1 amino acid sequence
GFTFSSYD

SEQ ID NO: 6 - protein, 8 aa, Artificial Sequence
  Synthetic 1F5 VH CDR2 amino acid sequence
IWYDGSNK

SEQ ID NO: 7 - protein, 12 aa, Artificial Sequence
  Synthetic 1F5 VH CDR3 amino acid sequence
ARGSGNWGFFDY

SEQ ID NO: 8 - protein, 6 aa, Artificial Sequence
  Synthetic 1F5 VL CDR1 amino acid sequence
QGISRW

SEQ ID NO: 9 - protein, 3 aa, Artificial Sequence
  Synthetic 1F5 VL CDR2 amino acid sequence
AAS

SEQ ID NO: 10 - protein, 9 aa, Artificial Sequence
  Synthetic 1F5 VL CDR3 amino acid sequence
QQYNTYPRT

SEQ ID NO: 11 - protein, 8 aa, Gallus gallus
  misc_feature: Ovalbumin peptide
SIINFEKL

SEQ ID NO: 12 - protein, 116 aa, Artificial Sequence
  Synthetic 2C2 VH amino acid sequence
QVQLVESGGGVVQPGRSLRLSCAASGFTFSSYDIHWVRQAPGKGLEWVAVIWNDGSNKYYADSVKGRFTISRDNSTNSLFLQMNSLRAEDTAVYYCVGGTADLEHWDQGTLVTVSS

SEQ ID NO: 13 - protein, 107 aa, Artificial Sequence
  Synthetic 2C2 VL amino acid sequence
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 14 - protein, 119 aa, Artificial Sequence
  Synthetic 3H12 VH amino acid sequence
QVQLVESGGGVVQPGRSLRLSCATSGFTFSSYDMHWVRQAPGKGLEWVAVIWYDGSNKYYADSVKGRFTISRDNSKNTLYLQMNSLGDEDTAVYYCARGSGNWGFFDYWGQGTLVTVSS

SEQ ID NO: 15 - protein, 107 aa, Artificial Sequence; identical to SEQ ID NO: 4
  Synthetic 3H12 VL amino acid sequence

SEQ ID NO: 16 - protein, 116 aa, Artificial Sequence
  Synthetic 2G9 VH amino acid sequence
QVQLVESGGGVVQPGRSLRLSCAASGFTLSSHDIHWVRQAPGKGLEWVAVIWNDGSNKYYADSVKGRFTISRDNSTNSLFLQMNSLRAEDTAVYYCVRGTADLEHWDQGTLVTVSS

SEQ ID NO: 17 - protein, 107 aa, Artificial Sequence; identical to SEQ ID NO: 13
  Synthetic 2G9 VL amino acid sequence

SEQ ID NO: 18 - protein, 118 aa, Artificial Sequence
  Synthetic 1H8 VH amino acid sequence
QVQLVESGGGVVQPGRSLRLSCAASGFTFNIYDMHWVRQAPGKGLEWVAVIWYDGSNQYYADSVKGRFTISRDNSKNTLYLQMNILRAEDTAVYYCARGTHWGYFDYWGQGTLVTVSS

SEQ ID NO: 19 - protein, 107 aa, Artificial Sequence
  Synthetic 1H8 VL amino acid sequence
DIQMTQSPSSLSASVGDRVTITCRASQGISSWLAWYQQKPEKAPKSLIYAASNLQSGVPSRFSGSGSGTDFTLTISSLQPEDFATYYCQQYNSYPRTFGQGTKVEIK

SEQ ID NO: 20 - protein, 124 aa, Artificial Sequence
  Synthetic 3A10 VH amino acid sequence
QVQLVESGGGVVQPGRSLRLSCAASGFTFSHYGMHWVRQAPGKGPEWVAIIWYDGSNKYYADSVKGRFTISRDNSKNTLDLQMNSLRAEDTAVYYCARDGWTTMVRGLNVFDIWGQGTMVTVSS

SEQ ID NO: 21 - protein, 107 aa, Artificial Sequence
  Synthetic 3A10 VL amino acid sequence
DIQMTQSPSSLSASVGDRVTITCRASQDISSWLAWYQQKPEKAPKSLIYAASSLQSGVPSRFSGSGSGTDFTLTISSLQPEDFATYYCQQYNSYPPTFGQGTRLEIK

SEQ ID NO: 22 - protein, 119 aa, Artificial Sequence
  Synthetic 3H8 VH amino acid sequence
EVQLVESGGGLVQPGGSLRLSCAASGFTFSSYWMAWVRQAPGKGLEWLGNIKQDGSEKYYVDSVKGRFTISRDNAKNSLYLQMNSLRAEDTAVYYCVRELGMDWYFDLWGRGTLVTVSS

SEQ ID NO: 23 - protein, 107 aa, Artificial Sequence
  Synthetic 3H8 VL amino acid sequence
EIVLTQSPATLSLSPGERATLSCRASQSVDSYLAWYQQKPGQAPRLLIYDASNRATGIPARFSGSGSGTDFTLTISNLEPEDFAVYYCQQRSNWPPTFGQGTKVEIK

SEQ ID NO: 24 - protein, 122 aa, Artificial Sequence
  Synthetic 1G5 VH amino acid sequence
QVQLVESGGGVVQPGRSLRLSCAASGFSFSSYGMHWVRQAPGKGLEWVALLWYDGSHKDFADSVKGRFTISRDNSKNTLDLQMNSLRAEDTAVYYCAREGLAVPGHWYFDLWGRGTLVTVSS

SEQ ID NO: 25 - protein, 107 aa, Artificial Sequence
  Synthetic 1G5 VL amino acid sequence
AIQLTQSPSSLSASVGDRVTITCRASQGISSALAWYQQKPGKAPKLLIYDASSLESGVPSRFSGSGSGTDFTLTISSLQPEDFATYYCQQFNTYPRTFGQGTKVEIK

SEQ ID NO: 26 - protein, 8 aa, Artificial Sequence
  Synthetic 2C2 VH CDR1 amino acid sequence
GFTFSSYD

SEQ ID NO: 27 - protein, 8 aa, Artificial Sequence
  Synthetic 2C2 VH CDR2 amino acid sequence
IWNDGSNK

SEQ ID NO: 28 - protein, 12 aa, Artificial Sequence
  Synthetic 2C2 VH CDR3 amino acid sequence
VGGTADLEHWDQ

SEQ ID NO: 29 - protein, 6 aa, Artificial Sequence
  Synthetic 2C2 VL CDR1 amino acid sequence
QGISSW

SEQ ID NO: 30 - protein, 3 aa, Artificial Sequence
  Synthetic 2C2 VL CDR2 amino acid sequence
AAS

SEQ ID NO: 31 - protein, 9 aa, Artificial Sequence
  Synthetic 2C2 VL CDR3 amino acid sequence
QQYNSYPLT

SEQ ID NO: 32 - protein, 8 aa, Artificial Sequence
  Synthetic 3H12 VH CDR1 amino acid sequence
GFTFSSYD

SEQ ID NO: 33 - protein, 8 aa, Artificial Sequence
  Synthetic 3H12 VH CDR2 amino acid sequence
IWYDGSNK

SEQ ID NO: 34 - protein, 12 aa, Artificial Sequence
  Synthetic 3H12 VH CDR3 amino acid sequence
ARGSGNWGFFDY

SEQ ID NO: 35 - protein, 6 aa, Artificial Sequence
  Synthetic 3H12 VL CDR1 amino acid sequence
QGISRW

SEQ ID NO: 36 - protein, 3 aa, Artificial Sequence
  Synthetic 3H12 VL CDR2 amino acid sequence
AAS

SEQ ID NO: 37 - protein, 9 aa, Artificial Sequence
  Synthetic 3H12 VL CDR3 amino acid sequence
QQYNTYPRT

SEQ ID NO: 38 - protein, 8 aa, Artificial Sequence
  Synthetic 2G9 VH CDR1 amino acid sequence
GFTLSSHD

SEQ ID NO: 39 - protein, 8 aa, Artificial Sequence
  Synthetic 2G9 VH CDR2 amino acid sequence
IWNDGSNK

SEQ ID NO: 40 - protein, 12 aa, Artificial Sequence
  Synthetic 2G9 VH CDR3 amino acid sequence
VRGTADLEHWDQ

SEQ ID NO: 41 - protein, 6 aa, Artificial Sequence
  Synthetic 2G9 VL CDR1 amino acid sequence
QGISSW

SEQ ID NO: 42 - protein, 3 aa, Artificial Sequence
  Synthetic 2G9 VL CDR2 amino acid sequence
AAS

SEQ ID NO: 43 - protein, 9 aa, Artificial Sequence
  Synthetic 2G9 VL CDR3 amino acid sequence
QQYNSYPLT

SEQ ID NO: 44 - protein, 8 aa, Artificial Sequence
  Synthetic 1H8 VH CDR1 amino acid sequence
GFTFNIYD

SEQ ID NO: 45 - protein, 8 aa, Artificial Sequence
  Synthetic 1H8 VH CDR2 amino acid sequence
IWYDGSNQ

SEQ ID NO: 46 - protein, 11 aa, Artificial Sequence
  Synthetic 1H8 VH CDR3 amino acid sequence
ARGTHWGYFDY

SEQ ID NO: 47 - protein, 6 aa, Artificial Sequence
  Synthetic 1H8 VL CDR1 amino acid sequence
QGISSW

SEQ ID NO: 48 - protein, 3 aa, Artificial Sequence
  Synthetic 1H8 VL CDR2 amino acid sequence
AAS

SEQ ID NO: 49 - protein, 9 aa, Artificial Sequence
  Synthetic 1H8 VL CDR3 amino acid sequence
QQYNSYPRT

SEQ ID NO: 50 - protein, 8 aa, Artificial Sequence
  Synthetic 3A10 VH CDR1 amino acid sequence
GFTFSHYG

SEQ ID NO: 51 - protein, 8 aa, Artificial Sequence
  Synthetic 3A10 VH CDR2 amino acid sequence
IWYDGSNK

SEQ ID NO: 52 - protein, 17 aa, Artificial Sequence
  Synthetic 3A10 VH CDR3 amino acid sequence
ARDGWTTMVRGLNVFDI

SEQ ID NO: 53 - protein, 6 aa, Artificial Sequence
  Synthetic 3A10 VL CDR1 amino acid sequence
QDISSW

SEQ ID NO: 54 - protein, 3 aa, Artificial Sequence
  Synthetic 3A10 VL CDR2 amino acid sequence
AAS

SEQ ID NO: 55 - protein, 9 aa, Artificial Sequence
  Synthetic 3A10 VL CDR3 amino acid sequence
QQYNSYPPT

SEQ ID NO: 56 - protein, 8 aa, Artificial Sequence
  Synthetic 3H8 VH CDR1 amino acid sequence
GFTFSSYW

SEQ ID NO: 57 - protein, 8 aa, Artificial Sequence
  Synthetic 3H8 VH CDR2 amino acid sequence
IKQDGSEK

SEQ ID NO: 58 - protein, 12 aa, Artificial Sequence
  Synthetic 3H8 VH CDR3 amino acid sequence
VRELGMDWYFDL

SEQ ID NO: 59 - protein, 6 aa, Artificial Sequence
  Synthetic 3H8 VL CDR1 amino acid sequence
QSVDSY

SEQ ID NO: 60 - protein, 3 aa, Artificial Sequence
  Synthetic 3H8 VL CDR2 amino acid sequence
DAS

SEQ ID NO: 61 - protein, 9 aa, Artificial Sequence
  Synthetic 3H8 VL CDR3 amino acid sequence
QQRSNWPPT

SEQ ID NO: 62 - protein, 8 aa, Artificial Sequence
  Synthetic 1G5 VH CDR1 amino acid sequence
GFSFSSYG

SEQ ID NO: 63 - protein, 9 aa, Artificial Sequence
  Synthetic 1G5 VH CDR2 amino acid sequence
LLWYDGSHK

SEQ ID NO: 64 - protein, 15 aa, Artificial Sequence
  Synthetic 1G5 VH CDR3 amino acid sequence
AREGLAVPGHWYFDL

SEQ ID NO: 65 - protein, 6 aa, Artificial Sequence
  Synthetic 1G5 VL CDR1 amino acid sequence
QGISSA

SEQ ID NO: 66 - protein, 3 aa, Artificial Sequence
  Synthetic 1G5 VL CDR2 amino acid sequence
DAS

SEQ ID NO: 67 - protein, 9 aa, Artificial Sequence
  Synthetic 1G5 VL CDR3 amino acid sequence
QQFNTYPRT

SEQ ID NO: 68 - protein, 452 aa, Artificial Sequence
  Synthetic Varlilumab Heavy Chain
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSS

SEQ ID NO: 69 - protein, 214 aa, Artificial Sequence
  Synthetic Varlilumab Light Chain
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 260 aa, Artificial Sequence
  Synthetic CD27R87A
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACANGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 71 - protein, 240 aa, Artificial Sequence
  Synthetic CD27R87A
TPAPKSCPERHYWAQGKLCCQMCEPGTFLVKDCDQHRKAAQCDPCIPGVSFSPDHHTRPHCESCRHCNSGLLVRNCTITANAECACANGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP